(19) United States
(12) Patent Application Publication
(10) Pub. No.: US 2020/0163644 A1
(43) Pub. Date: May 28, 2020

(54) FETAL HEART RATE MONITORING SYSTEM

(71) Applicant: Hera Med Ltd., Netanya (IL)

(72) Inventors: David GROBERMAN, Even -Yehuda (IL); Tal SLONIM, Rishon Lezion (IL); Shimon HAYUN, Rosh Haayin (IL); Joel ROTEM, Kfar-Sirkin (IL); Michael NENNER, Tel-Aviv (IL)

(73) Assignee: Hera Med Ltd., Netanya (IL)

(21) Appl. No.: 16/778,266

(22) Filed: Jan. 31, 2020

Related U.S. Application Data
(63) Continuation of application No. 14/917,968, filed on Mar. 10, 2016, now abandoned.
(60) Provisional application No. 61/875,722, filed on Sep. 10, 2013.

(51) Int. Cl. A61B 8/08; A61B 8/00; A61B 8/02

(52) U.S. Cl. CPC A61B 8/0866; A61B 8/543; A61B 8/488; A61B 5/02411; A61B 8/54; A61B 8/461; A61B 8/4254; A61B 8/02

(57) ABSTRACT

A fetal heart rate monitor useful for locating and monitoring fetal heartbeat signal is disclosed. The fetal heart rate monitor may include: at least one Doppler transducer for acquiring said fetal heartbeat signal; at least one processor configured for processing data received from said at least one Doppler transducer and for determining whether the data is sufficient for acquiring and monitoring the fetal heartbeat signal and, if not sufficient, for providing guidance to a user to make location changes; and, at least one communication module for providing the guidance to the user. Said guidance may include guiding instructions to detect said fetal heartbeat signal according to feedback received from said at least one Doppler transducer and processed by said at least one processor and may further include verbal instructions comprising directions where to move the Doppler transducer on an abdomen of an expected mother.

### CROSS-REFERENCE TO RELATED APPLICATIONS

This application is a continuation of U.S. patent application Ser. No. 14/917,968, filed Mar. 10, 2016, which is a National Phase of International Application No. PCT/IL2014/050730, filed Aug. 14, 2014, claiming priority from U.S. Provisional Patent Application No. 61/875,722, filed Sep. 10, 2013, all of which are hereby incorporated by reference.

### FIELD OF THE INVENTION

The present invention generally relates to an ultrasound Doppler fetal heart rate monitor (FHRM) device for receiving fetal heart rate (FHR) readings. More particularly, the present invention pertains to an FHRM intended for safe home-use which has technological solutions for simplifying the location of a fetal heartbeat (FHB) signal.

### BACKGROUND OF THE INVENTION

The Doppler fetal heart rate monitor (FHRM) is a device including a hand held ultrasound transducer used to detect and measure the heartbeat of a fetus for prenatal care. It uses the Doppler effect to provide an audible simulation of the fetal heartbeat (FHB). The monitor is placed on the expecting mother's abdomen. The device produces an ultrasonic beam, typically in the frequency range of 1-3 MHz, originating from the transducer. The ultrasonic beam reflected from the abdomen is received by the transducer and typically translated to a sound wave in the audible range. If the fetus heart is within the ultrasonic beam, movement of the fetus heart valves or its blood flow may be translated into an audible heart rate beat. Some systems further translate the FHB sound into a digital numeric reading corresponding to the fetal heartrate (FHR) in beats per minute (BPM). Originally intended for use by health care professionals, this device is becoming popular for personal use.

To date, Doppler FHRM share marketed for home use are difficult for anon-professional person to use since the device will produce no usable reading unless the transducer is placed in the precise location that will enable a typically narrow bandwidth beam to properly reflect the fetal heart valves. Locating the heartbeat requires practice, experience and former knowledge. Typically, location of the FHR requires placing the sensor within 1-2 cm of the optimal location. Often when an expectant mother tries to locate with a home use FHRM the heartbeat of her unborn child she has little success resulting in no heartbeat reading. Furthermore, the searching procedure is usually accompanied by unclear, unpleasant, and even stressful “white” sounds accompanied by other sounds which are produced by the device movement, the expecting mother's biologically oriented sounds (such as blood flow, stomach etc.) and the fetus's own movements. It is difficult for a non-trained user to understand and distinguish what he hears and whether it is the fetus heartbeat or not. The expectant mother has no way to know whether the lack of FHB signal is the result of improper use of the device or a medical problem with the fetus. The result is that instead of assuring the mother that her fetus is doing well, the mother is subjected to psychological stress that may be harmful for both her and the fetus. In addition, visits to physicians are needlessly increased.

Attempts to improve FHRM have been made. U.S. Pat. No. 5,827,969 recites a detection system that uses one transducer which has variable power settings for manually controlling the level of ultrasonic energy to increase sensitivity of the probe. In another example U.S. Pat. No. 6,551,251 recites an FHRM enabling to receive a good signal of the FHB regardless of its position in the womb. The monitor uses one frequency of the transducer to receive a signal from the fetus when it is in direct contact with the abdominal wall and a second frequency to receive a signal when the fetus is not in direct contact.

Additional disclosed solutions include using a plurality of transducers for increasing the effective area of coverage of the ultrasound probe (patent application US201 1/0160591) and applying all kind of filtering methods to increase the signal to noise ratio (for example, U.S. Pat. No. 5,524,631).

Therefore, there is a long felt and unmet need for new technologies that will make Doppler monitors for home use easier and safer to use without significantly increasing their cost.

### SUMMARY OF THE INVENTION

The present invention provides a fetal heart rate monitor (FHRM) (100) useful for locating fetal heartbeat (FHB) and monitoring the fetal heart rate (400), the FHRM comprising: at least one Doppler transducer (101); at least one processor (102); and at least one communication module (103); wherein the FHRM is operative in a method of: (a) obtaining an FHRM comprising at least one Doppler transducer; at least one Doppler transducer comprises a high frequency mode and a low frequency mode (401); (b) placing at least one Doppler transducer around the abdomen of an expectant mother (402); (c) setting at least one Doppler transducer to the low frequency mode; the low frequency mode has a wide beam that facilitates the location of the FHB (403); (d) moving at least one Doppler transducer past the abdomen until a location where the FHB signal is found (404); (e) switching at least one Doppler transducer to the high frequency mode whilst keeping at least one Doppler transducer in the location for receiving the fetal heartbeat (FHB) signal; the high frequency has a narrow beam adapted for concentrating and receiving accurate fetal heart rate (FHR) readings resulting in improved signal to noise ratio (405); and (f) detecting by means of the FHRM the FHB signals (406).

It is another object of the current invention to disclose the FHRM as defined in any of the above, wherein the switching is automatic.

It is another object of the current invention to disclose the FHRM as defined in any of the above, wherein the automatic switching from the low frequency mode to the high frequency mode occurs when the FHRM recognizes a signal sufficient to sustain accurate measurement of the FHR.

It is another object of the current invention to disclose the FHRM as defined in any of the above, wherein the FHRM recognizes the FHB signal by detecting a specific pattern of signals.

It is another object of the current invention to disclose the FHRM as defined in any of the above, wherein the method additionally comprises a step of switching back to the low frequency mode when the FHB signal is lost.

It is another object of the current invention to disclose the FHRM as defined in any of the above, wherein the method additionally comprises a step of guiding the user to make small location changes of at least one Doppler transducer when the FHB signal is lost when switching from the low frequency beam to the high frequency beam.

It is another object of the current invention to disclose the FHRM as defined in any of the above, wherein the method additionally comprises a step of turning the FHRM on; at least one Doppler transducer is in the low frequency mode when turned on.

It is another object of the current invention to disclose the FHRM as defined in any of the above, wherein the switching is semi-automatic.

It is another object of the current invention to disclose the FHRM as defined in any of the above, wherein the switching is manual.

It is another object of the current invention to disclose the FHRM as defined in any of the above, wherein the manual switching from the low frequency mode to the high frequency mode occurs when applying pressure to at least one Doppler transducer.

It is another object of the current invention to disclose the FHRM as defined in any of the above, wherein the manual switching is carried out in a manner selected from a group consisting of: (a) pushing a button; (b) moving a switch; and any combination thereof.

It is another object of the current invention to disclose the FHRM as defined in any of the above, wherein the method additionally comprises a step of notifying the user when the signal is sufficient to sustain accurate measurement.

It is another object of the current invention to disclose the FHRM as defined in any of the above, wherein the step of notifying is done by signaling the user; the signaling method is selected from a group of signals consisting of: (a) audible; (b) visual; (c) sensible; and any combination thereof.

It is another object of the current invention to disclose the FHRM as defined in any of the above, wherein the method additionally comprises a step of identifying the speed of movement of at least one Doppler transducer, carried out by an accelerometer attached to at least one Doppler transducer.

It is another object of the current invention to disclose the FHRM as defined in any of the above, wherein the method additionally comprises a step of guiding the user regarding the movement of at least one Doppler transducer; the guiding is selected from a group consisting of: (a) direction of movement; (b) speed of movement; and any combination thereof.

It is another object of the current invention to disclose the FHRM as defined in any of the above, wherein the step of guiding is carried out in a manner selected from a group consisting of: (a) graphical; (b) audible (c) sensible; and any combination thereof.

It is another object of the current invention to disclose the FHRM as defined in any of the above, wherein the FHRM is designed for home use.

It is another object of the current invention to disclose the FHRM as defined in any of the above, wherein at least one Doppler transducer is comprised of more than two frequency modes.

It is another object of the current invention to disclose the FHRM as defined in any of the above, wherein the method additionally comprises a step of gradually moving from the low frequency mode to the high frequency mode through at least one intermediate frequency.

It is another object of the current invention to disclose the FHRM as defined in any of the above, wherein the step of gradually moving from the low frequency mode to the high frequency mode is carried out continuously.

It is another object of the current invention to disclose the FHRM as defined in any of the above, wherein the method additionally comprises a step of transforming the FHB signal to an indication selected from a group consisting of: (a) audible; (b) visual; (c) sensible; and any combination thereof.

It is another object of the current invention to disclose the FHRM as defined in any of the above, wherein the processor and the communicating module is a one computerized device selected from a group consisting of: (a) mobile phone; (b) tablet; (c) laptop; (d) desktop; and any combination thereof.

It is another object of the current invention to disclose the FHRM as defined in any of the above, wherein the method additionally comprises a step of saving previous searches by the processor.

It is another object of the current invention to disclose the FHRM as defined in any of the above, wherein the method additionally comprises a step of utilizing the previously saved searches for facilitating new searches.

It is another object of the current invention to disclose the FHRM as defined in any of the above, wherein the communicating module is a GUI.

It is another object of the current invention to disclose the FHRM as defined in any of the above, wherein the method additionally comprises a step of displaying a map of the expectant mother's abdomen on the GUI; the map is capable of showing the location of the transducer and the direction at least on Doppler transducer should be moved.

It is another object of the current invention to disclose the FHRM as defined in any of the above, wherein the method additionally comprises a step of setting the high frequency beam to about 2-5 Mhz.

It is another object of the current invention to disclose the FHRM as defined in any of the above, wherein the method additionally comprises a step of setting the low frequency beam to about 200-500 KHz.

It is another object of the current invention to disclose the FHRM as defined in any of the above, wherein the Doppler transducer is comprised of at least one Piezoelectric transducer.

It is another object of the current invention to disclose the FHRM as defined in any of the above, wherein the low frequency beam and the high frequency beam are obtained from a single Piezoelectric transducer; the low frequency is the radial frequency and the high frequency is the natural frequency.

It is another object of the present invention to disclose a fetal heart rate monitor (FHRM) useful for locating fetal heartbeat (FHB) and monitoring the fetal heart rate (800), the FHRM comprising: at least one Doppler transducer; at least one processor; and at least one communication module; wherein the FHRM is operative in a method of: (a) obtaining an FHRM comprising at least one Doppler transducer; at least one Doppler transducer comprises a high intensity mode and a low intensity mode (80 I); (b) placing at least one Doppler transducer on the abdomen of an expectant mother (802); (c) setting at least one Doppler transducer to the high intensity mode: the high intensity mode facilitates the location of the FHB signal (803); (d) moving at least one Doppler transducer around the abdomen until a location where an FHB signal is found (804); (e) switching at least one Doppler transducer to the low intensity mode whilst keeping at least one Doppler transducer in the location for receiving the FHB signal; the low intensity mode allows monitoring the fetal heart rate (FHR) while only relatively small amount of energy is transmitted to the body of the expectant mother and the fetus and lengthening the battery life of at least one Doppler transducer (805); and (f) detecting by means of the FHRM the FHB signals (806).

It is another object of the current invention to disclose the FHRM as defined in any of the above, wherein the switching is automatic.

It is another object of the current invention to disclose the FHRM as defined in any of the above, wherein the automatic switching from the high intensity mode to the low intensity mode occurs when the FHRM recognizes a signal sufficient to sustain accurate measurement of the FHR.

It is another object of the current invention to disclose the FHRM as defined in any of the above, wherein the FHRM recognizes the FHB signal by detecting a specific pattern of signals.

It is another object of the current invention to disclose the FHRM as defined in any of the above, wherein the method additionally comprises a step of switching back to the high intensity mode when the FHB signal is lost.

It is another object of the current invention to disclose the FHRM as defined in any of the above, wherein the method additionally comprises a step of guiding the user to make small location changes of at least one Doppler transducer when the FHB signal is lost when switching from the high intensity beam to the low intensity beam.

It is another object of the current invention to disclose the FHRM as defined in any of the above, wherein the method additionally comprises a step of turning the FHRM on; the FHRM is set to the high intensity mode when turned on.

It is another object of the current invention to disclose the FHRM as defined in any of the above, wherein the switching is semi-automatic.

It is another object of the current invention to disclose the FHRM as defined in any of the above, wherein the switching is manual.

It is another object of the current invention to disclose the FHRM as defined in any of the above, wherein the manual switching from the high intensity mode to the low intensity mode occurs when applying pressure to at least one Doppler transducer.

It is another object of the current invention to disclose the FHRM as defined in any of the above, wherein the manual switching is carried out in a manner selected from a group consisting of: (a) pushing a button; (b) moving a switch; and any combination thereof.

It is another object of the current invention to disclose the FHRM as defined in any of the above, wherein the method additionally comprises a step of notifying the user when the sufficient signal to sustain accurate measurement of the FHR is found.

It is another object of the current invention to disclose the FHRM as defined in any of the above, wherein the step of notifying is done by signaling the user; the signaling method is selected from a group of signals consisting of: (a) audible; (b) visual; (c) sensible; and any combination thereof.

It is another object of the current invention to disclose the FHRM as defined in any of the above, wherein the method additionally comprises a step of identifying the speed of movement of at least one Doppler transducer carried out by an accelerometer attached to at least one Doppler transducer.

It is another object of the current invention to disclose the FHRM as defined in any of the above, wherein the method additionally comprises a step of guiding the user regarding the movement of at least one Doppler transducer; the guiding is selected from a group consisting of: (a) direction of movement; (b) speed of movement; and any combination thereof.

It is another object of the current invention to disclose the FHRM as defined in any of the above, wherein the step of guiding is carried out in a manner selected from a group consisting of: (a) graphical; (b) audible; (c) sensible; and any combination thereof.

It is another object of the current invention to disclose the FHRM as defined in any of the above, wherein the FHRM is designed for home use.

It is another object of the current invention to disclose the FHRM as defined in any of the above, wherein at least one Doppler transducer is comprised of more than two intensity modes.

It is another object of the current invention to disclose the FHRM as defined in any of the above, wherein the method additionally comprises a step of gradually moving from the high intensity mode to the low intensity mode through at least one intermediate intensity.

It is another object of the current invention to disclose the FHRM as defined in any of the above, wherein the step of gradually moving from the high intensity mode to the low intensity mode is carried out continuously.

It is another object of the current invention to disclose the FHRM as defined in any of the above, wherein the method additionally comprises a step of gradually moving back from the low intensity mode back to the high intensity mode when the FHB signal is lost until reaching the intensity in which the signal is re-discovered.

It is another object of the current invention to disclose the FHRM as defined in any of the above, wherein the method additionally comprises a step of transforming the FHB signal to an indication selected from a group consisting of: (a) audible; (b) visual; (c) sensible; and any combination thereof.

It is another object of the current invention to disclose the FHRM as defined in any of the above, wherein the processor and the communicating module is a one computerized device selected from a group consisting of: (a) mobile phone; (b) tablet; (c) laptop; (d) desktop; and any combination thereof.

It is another object of the current invention to disclose the FHRM as defined in any of the above, wherein the method additionally comprises a step of saving previous searches by the processor.

It is another object of the current invention to disclose the FHRM as defined in any of the above, wherein the method additionally comprises a step of utilizing the previously saved searches for facilitating new searches.

It is another object of the current invention to disclose the FHRM as defined in any of the above, wherein the communicating module is a GUI.

It is another object of the current invention to disclose the FHRM as defined in any of the above, wherein the method additionally comprises a step of displaying a map of the expectant mother's abdomen on the GUI; the map is capable of showing the location of the transducer and the direction at least on Doppler transducer should be moved.

It is another object of the current invention to disclose the FHRM as defined in any of the above, wherein the high intensity beam is about 100 mW/cm2.

It is another object of the current invention to disclose the FHRM as defined in any of the above, wherein the low intensity beam is about 10 mW/cm2.

It is another object of the current invention to disclose a fetal heart rate monitor (FHRM) useful for locating and monitoring fetal heartbeat (FHB), the FHRM, comprising: (a) at least one Doppler transducer comprising at least one Piezoelectric ceramic element placeable on an abdomen of an expectant mother for acquiring the FHB signal; (b) at least one pressure sensing module; (c) at least one processor for processing data received from at least one Doppler transducer and at least one pressure sensing module; and (d) at least one communication module for transmitting the processed data to a user; wherein at least one pressure sensing module and at least one transducer are operably coupled further wherein at least one pressure sensing module is adapted to detect pressure of the abdomen on at least one Doppler transducer.

It is another object of the current invention to disclose the FHRM as defined in any of the above, wherein at least one pressure sensing module additionally senses the directionality of the pressure.

It is another object of the current invention to disclose the FHRM as defined in any of the above, wherein at least one sensing module is made of a plurality of pressure sensors; the plurality of pressure sensors are located across the surface of at least one Doppler transducer which is in direct contact with the abdomen of an expectant mother.

It is another object of the current invention to disclose the FHRM as defined in any of the above, wherein the plurality of pressure sensors are located around the rim of at least one Doppler transducer which is in direct contact with the abdomen of an expectant mother.

It is another object of the current invention to disclose the FHRM as defined in any of the above, wherein the processor calculates the center of pressure according to the pressure in each of the plurality of sensors.

It is another object of the current invention to disclose the FHRM as defined in any of the above, wherein the processor calculates the directionality of the pressure according to the pressure in each of the plurality of sensors.

It is another object of the current invention to disclose the FHRM as defined in any of the above, wherein at least one communication module alerts the user when not enough pressure is applied on at least one Doppler transducer.

It is another object of the current invention to disclose the FHRM as defined in any of the above, wherein the alert is carried out in a manner selected from a group consisting of: (a) audible; (b) sensible; (c) visual; and any combination thereof.

It is another object of the current invention to disclose the FHRM as defined in any of the above, wherein at least one processor turns on at least one transducer only when enough pressure is applied on at least one transducer to detect the FHB signal.

It is another object of the current invention to disclose the FHRM as defined in any of the above, wherein at least one processor turns off at least one transducer when not enough pressure is applied on at least one transducer to detect the FHB signal.

It is another object of the current invention to disclose the FHRM as defined in any of the above, wherein at least one communication module notifies the user on the directionality of pressure in which the ideal FHB signal is acquired.

It is another object of the current invention to disclose the FHRM as defined in any of the above, wherein the communication module notifies in a manner selected from a group consisting of: (a) audible; (b) sensible; (c) visual; and any combination thereof.

It is another object of the current invention to disclose the FHRM as defined in any of the above, wherein at least one communication module gives directions to the user to move at least one transducer in a direction opposite to the directionality of the pressure in which the ideal FHB signal is acquired.

It is another object of the current invention to disclose the FHRM as defined in any of the above, wherein the directions are given in a manner selected from a group consisting of: (a) audible; (b) sensible; (c) visual; and any combination thereof.

It is another object of the current invention to disclose the FHRM as defined in any of the above, wherein at least one communication module is a computerized device selected from a group consisting of: (a) smartphone; (b) tablet; (c) laptop; (d) desktop; and any combination thereof.

It is another object of the current invention to disclose the FHRM as defined in any of the above, wherein the FHRM is designed for home use or for ambulatory care.

It is another object of the current invention to disclose the FHRM as defined in any of the above, wherein at least one processor and at least one communicating module is a one computerized device selected from a group consisting of: (a) mobile phone (b) smartphone; (b) tablet; (c) laptop; (d) desktop; and any combination thereof.

It is another object of the current invention to disclose the FHRM as defined in any of the above, wherein previous searches can be saved.

It is another object of the current invention to disclose the FHRM as defined in any of the above, wherein the saved previous searches can be utilized for facilitating new searches.

It is another object of the current invention to disclose the FHRM as defined in any of the above, wherein the communicating module is a GUI.

It is another object of the current invention to disclose the FHRM as defined in any of the above, wherein the GUI displays a map or an illustration of the expectant mother's abdomen; the map is capable of showing the location of at least one Doppler transducer and the direction at least on Doppler transducer should be moved.

It is another object of the current invention to disclose the FHRM as defined in any of the above, wherein the pressure sensing module is at least one Piezoelectric ceramic element of at least one Doppler transducer.

It is another object of the current invention to disclose the FHRM as defined in any of the above, wherein at least one Piezoelectric ceramic element detects pressure by changes in its electric capacity as a result of the pressure; the electric capacity increases as the pressure increases.

It is another object of the current invention to disclose the FHRM as defined in any of the above, wherein at least one Doppler transducer contains a plurality of Piezoelectric ceramic elements.

It is another object of the current invention to disclose the FHRM as defined in any of the above, wherein the pressure sensing module is the plurality of Piezoelectric ceramic elements of at least one Doppler transducer.

It is another object of the current invention to disclose the FHRM as defined in any of the above, wherein the plurality of Piezoelectric ceramic elements additionally senses the directionality of the pressure.

It is another object of the current invention to disclose a fetal heart rate monitor (FHRM) useful for locating and monitoring fetal heartbeat (FHB), the FHRM, comprising: (a) at least one Doppler transducer comprising at least one Piezoelectric ceramic element 110 placeable on an abdomen of an expectant mother for acquiring the FHB signal; (b) at least one processor 130 for processing data received from at least one Doppler transducer; and (c) at least one communication module for transmitting the processed data to a user, wherein at least one Piezoelectric ceramic element is further adapted to detect pressure of the abdomen on at least one Doppler transducer.

It is another object of the current invention to disclose the FHRM as defined in any of the above, wherein at least one Piezoelectric ceramic element detects pressure by changes in its electric capacity as a result of the pressure; the electric capacity increases as the pressure increases.

It is another object of the current invention to disclose the FHRM as defined in any of the above, wherein at least one Doppler transducer contains a plurality of Piezoelectric ceramic elements.

It is another object of the current invention to disclose the FHRM as defined in any of the above, wherein the plurality of Piezoelectric ceramic elements additionally detects the directionality of the pressure.

It is another object of the current invention to disclose the FHRM as defined in any of the above, wherein the processor calculates the center of pressure according to the electric capacity in each of the plurality of the plurality of Piezoelectric ceramic elements.

It is another object of the current invention to disclose the FHRM as defined in any of the above, wherein the processor calculates the directionality of the pressure according to the electric capacity in each of the plurality of Piezoelectric ceramic elements.

It is another object of the current invention to disclose the FHRM as defined in any of the above, wherein at least one communication module alerts the user when not enough pressure is applied on at least one transducer.

It is another object of the current invention to disclose the FHRM as defined in any of the above, wherein the alert is carried out in a manner selected from a group consisting of: (a) audible; (b) sensible; (c) visual; and any combination thereof.

It is another object of the current invention to disclose the FHRM as defined in any of the above, wherein at least one processor turns off at least one transducer when not enough pressure is applied on at least one transducer to detect the FHB signal.

It is another object of the current invention to disclose the FHRM as defined in any of the above, wherein at least one processor turns on at least one transducer only when enough pressure is applied on at least one transducer to detect the FHB signal.

It is another object of the current invention to disclose the FHRM as defined in any of the above, wherein at least one communication module alerts the user on the directionality of pressure in which the ideal FHB signal is acquired.

It is another object of the current invention to disclose the FHRM as defined in any of the above, wherein at least one communication module gives directions to the user to move at least one transducer in a direction opposite to the directionality of the pressure in which the ideal FHB signal is acquired.

It is another object of the current invention to disclose the FHRM as defined in any of the above, wherein the directions are given in a manner selected from a group consisting of: (a) audible; (b) sensible; (c) visual; and any combination thereof.

It is another object of the current invention to disclose the FHRM as defined in any of the above, wherein the communication module is a computerized device selected from a group consisting of: (a) mobile phone; (b) smartphone; (c) tablet; (d) laptop; (e) desktop; and any combination thereof.

It is another object of the current invention to disclose the FHRM as defined in any of the above, wherein the FHRM is designed for home use or for ambulatory care.

It is another object of the current invention to disclose the FHRM as defined in any of the above, wherein the processor and the communicating module is a one computerized device selected from a group consisting of: (a) mobile phone; (b) smartphone (c) tablet; (d) laptop; (e) desktop; and any combination thereof.

It is another object of the current invention to disclose the FHRM as defined in any of the above, wherein previous searches can be saved.

It is another object of the current invention to disclose the FHRM as defined in any of the above, wherein the saved previous searches can be utilized for facilitating new searches.

It is another object of the current invention to disclose the FHRM as defined in any of the above, wherein the communicating module is a GUI.

It is another object of the current invention to disclose the FHRM as defined in any of the above, wherein the GUI displays a map or an illustration of the expectant mother's abdomen; the map is capable of showing the location of at least one Doppler transducer and the direction at least one Doppler transducer should be moved.

It is another object of the current invention to disclose a processor for operating a Doppler transducer, placeable on an abdomen of an expectant mother, useful for locating and monitoring fetal heartbeat (FHB), the processor, comprising: (a) an input module for receiving data of the FHB signal generated by the Doppler transducer; (b) an analyzing module for analyzing the data; and (c) an output module for transmitting analyzed data to a user; wherein the data additionally includes the amount of pressure of the abdomen of an expectant mother on the Doppler transducer.

It is another object of the current invention to disclose the FHRM as defined in any of the above, wherein the data additionally includes directionality of the amount of pressure.

It is another object of the current invention to disclose the FHRM as defined in any of the above, wherein the analyzing module analyzes threshold pressure from which the FHB signal will be acquired.

It is another object of the current invention to disclose the FHRM as defined in any of the above, wherein the output module alerts the user when not enough pressure is applied on the Doppler transducer.

It is another object of the current invention to disclose the FHRM as defined in any of the above, wherein the alert is carried out in a manner selected from a group consisting of: (a) audible; (b) sensible; (c) visual; and any combination thereof.

It is another object of the current invention to disclose the FHRM as defined in any of the above, wherein the analyzing module turns on the Doppler transducer only when enough pressure is applied to it to detect the FHB signal.

It is another object of the current invention to disclose the FHRM as defined in any of the above, wherein the analyzing module turns off the Doppler transducer when not enough pressure is applied to it to detect the FHB signal.

It is another object of the current invention to disclose the FHRM as defined in any of the above, wherein the output module notifies the user on the directionality of pressure in which the ideal FHB signal is acquired.

It is another object of the current invention to disclose the FHRM as defined in any of the above, wherein the output module notifies in a manner selected from a group consisting of: (a) audible; (b) sensible; (c) visual; and any combination thereof.

It is another object of the current invention to disclose the FHRM as defined in any of the above, wherein the output module directs the user to move the Doppler transducer in a direction opposite to the directionality of the pressure in which the ideal FHB signal is acquired. The processor of claim 56, wherein the directions are given in a manner selected from a group consisting of: (a) audible; (b) sensible; (c) visual; and any combination thereof.

It is another object of the current invention to disclose the FHRM as defined in any of the above, wherein at least one of the input module, analyzing module and output module are a computerized device selected from a group consisting of: (a) mobile phone; (b) smartphone; (c) tablet; (d) laptop; (e) desktop; and any combination thereof.

It is another object of the current invention to disclose the FHRM as defined in any of the above, additionally comprising a logging module.

It is another object of the current invention to disclose the FHRM as defined in any of the above, wherein previous searches can be saved.

It is another object of the current invention to disclose a guiding module, useful for guiding a user to locate a fetal heartbeat (FHB) signal with a Doppler transducer operably coupled to a plurality of pressure sensors, comprising: (a) at least one processor for processing data received from at least one Doppler transducer and the plurality of pressure sensors; and (b) at least one communication module for transmitting the processed data to a user; wherein the plurality of pressure sensors measure the pressure of the Doppler transducer on the abdomen of an expectant mother and at least one processor calculates the directionality of the pressure in which the ideal FHB signal is acquired; further wherein the communicating module guides the user to move at least one Doppler transducer in a direction opposite to the directionality.

It is another object of the current invention to disclose the FHRM as defined in any of the above, wherein the processor calculates the directionality of the pressure according to the pressure in each of the plurality of sensors.

It is another object of the current invention to disclose the FHRM as defined in any of the above, wherein at least one communication module alerts the user when not enough pressure is applied on the Doppler transducer.

It is another object of the current invention to disclose the FHRM as defined in any of the above, wherein the alert is carried out in a manner selected from a group consisting of: (a) audible; (b) sensible; (c) visual; and any combination thereof.

It is another object of the current invention to disclose the FHRM as defined in any of the above, wherein the communication module guides in a manner selected from a group consisting of: (a) audible; (b) sensible; (c) visual; and any combination thereof.

It is another object of the current invention to disclose the FHRM as defined in any of the above, wherein at least one communication module is a computerized device selected from a group consisting of: (a) mobile phone; (b) smartphone; (c) tablet; (d) laptop; (e) desktop; and any combination thereof.

It is another object of the current invention to disclose the FHRM as defined in any of the above, wherein at least one processor and at least one communicating module is a one computerized device selected from a group consisting of: (a) mobile phone; (b) smartphone; (c) tablet; (d) laptop; (e) desktop; and any combination thereof.

It is another object of the current invention to disclose the FHRM as defined in any of the above, wherein previous searches can be saved.

It is another object of the current invention to disclose the FHRM as defined in any of the above, wherein the saved previous searches can be utilized for facilitating new searches.

It is another object of the current invention to disclose the FHRM as defined in any of the above, wherein the communicating module is a GUI.

It is another object of the current invention to disclose the FHRM as defined in any of the above, wherein the GUI displays a map or an illustration of the expectant mother's abdomen; the map is capable of showing the location of at least one Doppler transducer and the direction at least on Doppler transducer should be moved.

It is another object of the current invention to disclose a method for operating a fetal heart rate monitor (FHRM) useful for locating and monitoring fetal heartbeat (FHB), comprising steps of: (a) obtaining an FHRM with at least one Doppler transducer containing at least one Piezoelectric ceramic element, at least one pressure sensing module and at, least one processor and at least one communication module; (b) placing at least one Doppler transducer on the abdomen of an expectant mother; (c) moving at least one Doppler transducer around the abdomen of an expectant mother; and (d) searching for an FHB signal; wherein the step of obtaining an FHRM additionally comprises a step of operably coupling at least one transducer and at least one pressure sensing module and the step of searching additionally comprises a step of detecting the pressure of the abdomen on at least one Doppler transducer.

It is another object of the current invention to disclose the FHRM as defined in any of the above, additionally comprising a step of making at least one sensing module from a plurality of pressure sensors; the plurality of pressure sensors are located across the surface of at least one Doppler transducer which is in direct contact with the abdomen of an expectant mother.

It is another object of the current invention to disclose the FHRM as defined in any of the above, additionally comprising a step of sensing the directionality of the pressure by at least one pressure sensing module.

It is another object of the current invention to disclose the FHRM as defined in any of the above, additionally comprising a step of locating the plurality of pressure sensors around the rim of at least one Doppler transducer which is in direct contact with the abdomen of an expectant mother.

It is another object of the current invention to disclose the FHRM as defined in any of the above, additionally comprising a step of calculating the center of pressure according to the pressure in each of the plurality of sensors.

It is another object of the current invention to disclose the FHRM as defined in any of the above, additionally comprising a step of calculating the directionality of the pressure according to the pressure in each of the plurality of sensors.

It is another object of the current invention to disclose the FHRM as defined in any of the above, additionally comprising a step of alerting by at least one communication module, the user when not enough pressure is applied on at least one transducer.

It is another object of the current invention to disclose the FHRM as defined in any of the above, additionally comprising a step of selecting a manner in which the alerting is carried out from a group consisting of: (a) audible; (b) sensible; (c) visual; and any combination thereof.

It is another object of the current invention to disclose the FHRM as defined in any of the above, additionally comprising a step of turning on at least one transducer by at least one processor only when enough pressure is applied on at least one transducer to detect the FHB signal.

It is another object of the current invention to disclose the FHRM as defined in any of the above, additionally comprising a step of turning off at least one transducer by at least one processor when not enough pressure is applied on at least one transducer to detect the FHB signal.

It is another object of the current invention to disclose the FHRM as defined in any of the above, additionally comprising a step of notifying by the user by the communication module on the directionality of pressure in which the ideal FHB signal is acquired.

It is another object of the current invention to disclose the FHRM as defined in any of the above, additionally comprising a step of selecting a manner of notifying from a group consisting of: (a) audible; (b) sensible; (c) visual; and any combination thereof.

It is another object of the current invention to disclose the FHRM as defined in any of the above, additionally comprising a step of directing the user by the communication module to move at least one transducer in a direction opposite to the directionality of the pressure in which the ideal FHB signal is acquired.

It is another object of the current invention to disclose the FHRM as defined in any of the above, additionally comprising a step of selecting a manner of directing from a group consisting of: (a) audible; (b) sensible; (c) visual; and any combination thereof.

It is another object of the current invention to disclose the FHRM as defined in any of the above, additionally comprising a step of selecting at least one communication module from a group consisting of: (a) mobile phone; (b) smartphone; (c) tablet; (d) laptop; (e) desktop; and any combination thereof.

It is another object of the current invention to disclose the FHRM as defined in any of the above, additionally comprising a step of designing the FHRM is for home use.

It is another object of the current invention to disclose the FHRM as defined in any of the above, additionally comprising a step of making at least one processor and at least one communicating module as a one computerized device selected from a group consisting of: (a) mobile phone; (b) smartphone; (c) tablet; (d) laptop; (e) desktop; and any combination thereof.

It is another object of the current invention to disclose the FHRM as defined in any of the above, additionally comprising a step of saving previous searches.

It is another object of the current invention to disclose the FHRM as defined in any of the above, additionally comprising a step of utilizing the previously saved searches for facilitating new searches.

It is another object of the current invention to disclose the FHRM as defined in any of the above, additionally comprising a step of selecting the communicating module to be a GUI.

It is another object of the current invention to disclose the FHRM as defined in any of the above, additionally comprising a step of displaying a map or an illustration of the expectant mother's abdomen on the GUI; the map is capable of showing the location of the transducer and the direction at least on Doppler transducer should be moved.

It is another object of the current invention to disclose the FHRM as defined in any of the above, wherein the pressure sensing module is at least one Piezoelectric ceramic element of at least one Doppler transducer.

It is another object of the current invention to disclose the FHRM as defined in any of the above, additionally comprising a step of detecting pressure by changes in its electric capacity as a result of the pressure; the electric capacity increases as the pressure increases.

It is another object of the current invention to disclose the FHRM as defined in any of the above, wherein at least one Doppler transducer contains a plurality of Piezoelectric ceramic elements.

It is another object of the current invention to disclose the FHRM as defined in any of the above, additionally comprising a step of sensing directionality of the pressure.

It is another object of the current invention to disclose a method for operating a fetal heart rate monitor (FHRM) useful for locating and monitoring fetal heartbeat (FHB), comprising steps of: (a) obtaining an FHRM with at least one Doppler transducer containing at least one Piezoelectric ceramic element, at least one processor and at least one communication module; (b) placing at least one Doppler transducer on the abdomen of an expectant mother; (c) moving at least one Doppler transducer around the abdomen of an expectant mother; and (d) searching for an FHB signal; wherein additionally comprising a step of detecting the pressure of the abdomen on at least one Doppler transducer by at least one Piezoelectric ceramic element.

It is another object of the current invention to disclose the FHRM as defined in any of the above, additionally comprising a step of detecting by at least one Piezoelectric ceramic element by changes in its electric capacity as a result of the pressure; the electric capacity increases as the pressure increases.

It is another object of the current invention to disclose the FHRM as defined in any of the above, wherein at least one Doppler transducer contains a plurality of Piezoelectric ceramic elements.

It is another object of the current invention to disclose the FHRM as defined in any of the above, additionally comprising a step of detecting directionality of the pressure by the plurality of Piezoelectric ceramic elements.

It is another object of the current invention to disclose the FHRM as defined in any of the above, additionally comprising a step of calculating the center of pressure according to the electric capacity in each of the plurality of the plurality of Piezoelectric ceramic elements.

It is another object of the current invention to disclose the FHRM as defined in any of the above, additionally comprising a step of calculating the directionality of the pressure according to the electric capacity in each of the plurality of Piezoelectric ceramic elements.

It is another object of the current invention to disclose the FHRM as defined in any of the above, additionally comprising a step of alerting by at least one communication module the user when not enough pressure is applied on at least one transducer.

It is another object of the current invention to disclose the FHRM as defined in any of the above, additionally comprising a step of selecting a manner in which the alerting is carried out from a group consisting of: (a) audible; (b) sensible; (c) visual; and any combination thereof.

It is another object of the current invention to disclose the FHRM as defined in any of the above, additionally comprising a step of turning on at least one transducer by at least one processor only when enough pressure is applied on at least one transducer to detect the FHB signal.

It is another object of the current invention to disclose the FHRM as defined in any of the above, additionally comprising a step of turning off at least one transducer by at least one processor when not enough pressure is applied on at least one transducer to detect the FHB signal.

It is another object of the current invention to disclose the FHRM as defined in any of the above, additionally comprising a step of notifying by the user by the communication module on the directionality of pressure in which the ideal FHB signal is acquired.

It is another object of the current invention to disclose the FHRM as defined in any of the above, additionally comprising a step of selecting a manner of notifying from a group consisting of: (a) audible; (b) sensible; (c) visual; and any combination thereof.

It is another object of the current invention to disclose the FHRM as defined in any of the above, additionally comprising a step of directing the user by the communication module to move at least one transducer in a direction opposite to the directionality of the pressure in which the ideal FHB signal is acquired.

It is another object of the current invention to disclose the FHRM as defined in any of the above, additionally comprising a step of selecting a manner of directing from a group consisting of: (a) audible; (b) sensible; (c) visual; and any combination thereof.

It is another object of the current invention to disclose the FHRM as defined in any of the above, additionally comprising a step of selecting at least one communication module from a group consisting of: (a) mobile phone; (b) smartphone; (c) tablet; (d) laptop; (e) desktop; and any combination thereof.

It is another object of the current invention to disclose the FHRM as defined in any of the above, additionally comprising a step of designing the FHRM is for home use or for ambulatory care.

It is another object of the current invention to disclose the FHRM as defined in any of the above, additionally comprising a step of making at least one processor and at least one communicating module as a one computerized device selected from a group consisting of: (a) mobile phone; (b) smartphone; (c) tablet; (d) laptop; (e) desktop; and any combination thereof.

It is another object of the current invention to disclose the FHRM as defined in any of the above, additionally comprising a step of saving previous searches.

It is another object of the current invention to disclose the FHRM as defined in any of the above, additionally comprising a step of utilizing the previously saved searches for facilitating new searches.

It is another object of the current invention to disclose the FHRM as defined in any of the above, additionally comprising a step of selecting the communicating module to be a GUI.

It is another object of the current invention to disclose the FHRM as defined in any of the above, additionally comprising a step of displaying a map or an illustration of the expectant mother's abdomen on the GUI; the map is capable of showing the location of the transducer and the direction at least on Doppler transducer should be moved.

It is another object of the current invention to disclose a method for guiding a user to locate a fetal heartbeat (FHB) signal with a fetal heart rate monitor (FHRM), comprising steps of: (a) obtaining an FHRM comprising at least one Doppler transducer, a plurality of pressure sensors, at least one processor and at least one communication module; (b) placing at least one Doppler transducer on the abdomen of an expectant mother; (c) angling at least one Doppler transducer; (d) moving at least one Doppler transducer around the abdomen; and (e) searching for an FHB signal; wherein the step of angling additionally comprises steps of applying pressure to at least one Doppler transducer on the abdomen; measuring the pressure by the plurality of pressure sensors; and calculating the directionality of the pressures by at least one processor in which the FHB signal is acquired; further wherein additionally comprising a step of guiding the user by at least one communication module to move at least one Doppler transducer in a direction opposite to the directionality.

It is another object of the current invention to disclose the FHRM as defined in any of the above, additionally comprising a step of alerting by at least one communication module, the user when not enough pressure is applied on at least one transducer.

It is another object of the current invention to disclose the FHRM as defined in any of the above, additionally comprising a step of selecting a manner of guiding from a group consisting of: (a) audible; (b) sensible; (c) visual; and any combination thereof.

It is another object of the current invention to disclose the FHRM as defined in any of the above, additionally comprising a step of selecting at least one communication module from a group consisting of: (a) mobile phone; (b) smartphone; (c) tablet; (d) laptop; (e) desktop; and any combination thereof.

It is another object of the current invention to disclose the FHRM as defined in any of the above, additionally comprising a step of making at least one processor and at least one communicating module as a one computerized device selected from a group consisting of: (a) mobile phone; (b) smartphone (c) tablet; (d) laptop; (e) desktop; and any combination thereof.

It is another object of the current invention to disclose the FHRM as defined in any of the above, additionally comprising a step of saving previous searches.

It is another object of the current invention to disclose the FHRM as defined in any of the above, additionally comprising a step of utilizing the previously saved searches for facilitating new searches.

It is another object of the current invention to disclose the FHRM as defined in any of the above, additionally comprising a step of selecting the communicating module to be a GUI.

It is another object of the current invention to disclose the FHRM as defined in any of the above, additionally comprising a step of displaying a map or an illustration of the expectant mother's abdomen on the GUI; the map is capable of showing the location of the transducer and the direction at least on Doppler transducer should be moved.

It is another object of the current invention to disclose a fetal heart rate monitor (FHRM) useful for locating and monitoring fetal heartbeat (FHB) signal, the FHRM comprising: (a) at least one Doppler transducer for acquiring the FHB signal; (b) at least one processor for processing data received from at least one Doppler transducer; and (c) at least one communication module for transmitting the processed data to a user; wherein the communicating module comprises user guidance means for guiding the user towards the detection of the FHB signal according to feedback the data received from at least one Doppler transducer and processed by at least one processor.

It is another object of the current invention to disclose the FHRM as defined in any of the above, wherein at least one Doppler transducer is placeable on the abdomen of an expectant mother.

It is another object of the current invention to disclose the FHRM as defined in any of the above, wherein the FHRM is intended for home use or ambulatory care.

It is another object of the current invention to disclose the FHRM as defined in any of the above, wherein the user guidance means include general instructions for detection of the FHB signal.

It is another object of the current invention to disclose the FHRM as defined in any of the above, wherein the general instructions include initiating location of the FHB signal at the middle-top part of the abdomen of an expectant mother.

It is another object of the current invention to disclose the FHRM as defined in any of the above, wherein the general instructions are adjusted according to general medical knowledge.

It is another object of the current invention to disclose the FHRM as defined in any of the above, wherein the general instructions are adjusted according to the week of gestation.

It is another object of the current invention to disclose the FHRM as defined in any of the above, wherein the user guidance means facilitate user guidance regarding features selected from a group consisting of: (a) angling; (b) pressure of at least one Doppler transducer on the abdomen of an expectant mother; (c) direction of movement of at least one Doppler transducer on the abdomen of an expectant mother; (d) speed of movement of at least one Doppler transducer on the abdomen of an expectant mother; (e) quality of the FHB signal; and any combination thereof.

It is another object of the current invention to disclose the FHRM as defined in any of the above, wherein the user guidance means are selected from a group of consisting of: (a) audible; (b) sensible; (c) visual; and any combination thereof.

It is another object of the current invention to disclose the FHRM as defined in any of the above, wherein the audible guidance are beeping sounds.

It is another object of the current invention to disclose the FHRM as defined in any of the above, wherein the audible guidance are verbal instructions.

It is another object of the current invention to disclose the FHRM as defined in any of the above, wherein the visual guidance are flashing lights.

It is another object of the current invention to disclose the FHRM as defined in any of the above, wherein the visual guidance is a graphical map of the abdomen of an expectant mother.

It is another object of the current invention to disclose the FHRM as defined in any of the above, wherein the graphical map displays the approximate position of the fetus and the location of the fetus's heart.

It is another object of the current invention to disclose the FHRM as defined in any of the above, wherein the graphical map displays to the user the location of at least one Doppler transducer on the graphical map of the abdomen of an expectant mother on the graphical map.

It is another object of the current invention to disclose the FHRM as defined in any of the above, wherein the sensible guidance is based on haptic feedback.

It is another object of the current invention to disclose the FHRM as defined in any of the above, wherein the communicating module is a GUI selected from a group consisting of: (a) laptop; (b) smartphone; (c) computer; (d) tablet (e) mobile phone; and any combination thereof.

It is another object of the current invention to disclose the FHRM as defined in any of the above, wherein the GUI is a touch screen; the touch screen illustrates the graphical map of the abdomen of an expectant mother.

It is another object of the current invention to disclose the FHRM as defined in any of the above, wherein the user can notify at least one processor through the touch screen on the location of at least one transducer on the abdomen of an expectant mother.

It is another object of the current invention to disclose the FHRM as defined in any of the above, wherein the user can notify through the touch screen the location of at least one transducer on the abdomen of an expectant mother in which the FHB signal was detected

It is another object of the current invention to disclose the FHRM as defined in any of the above, wherein the GUI is controlled by eye movement.

It is another object of the current invention to disclose the FHRM as defined in any of the above, wherein the GUI is controlled by a pointing device.

It is another object of the current invention to disclose the FHRM as defined in any of the above, wherein the GUI is controlled by a mouse.

It is another object of the current invention to disclose the FHRM as defined in any of the above, wherein the location of at least one transducer on the abdomen of an expectant mother in which the FHB signal was detected can be utilized by at least one processor for future guiding towards the detection of the FHB signal.

It is another object of the current invention to disclose the FHRM as defined in any of the above, wherein at least one processor and the communicating module are a one computerized device selected from a group consisting of: (a) smartphone; (b) tablet; (c) laptop; (d) desktop; (e) mobile phone; and any combination thereof.

It is another object of the current invention to disclose the FHRM as defined in any of the above, wherein the user guidance is additionally based on data from previous searches of FHB signals.

It is another object of the current invention to disclose the FHRM as defined in any of the above, wherein the user guidance based on the previous searches takes into considerations the time passed from the previous searches.

It is another object of the current invention to disclose the FHRM as defined in any of the above, wherein the user guidance is based on the position of the fetus.

It is another object of the current invention to disclose the FHRM as defined in any of the above, wherein the user guidance gives indication when the FHB signal is found.

It is another object of the current invention to disclose the FHRM as defined in any of the above, wherein the indication is selected from a group of indications consisting of: (a) sensible; (b) audible, (c) visual.

It is another object of the current invention to disclose the FHRM as defined in any of the above, wherein the user guidance instructs the user not to move at least one Doppler transducer when the FHB signal is detected.

It is another object of the current invention to disclose the FHRM as defined in any of the above, wherein the user guidance alerts the user when the location of the FHB signal is not effective.

It is another object of the current invention to disclose the FHRM as defined in any of the above, wherein the user guidance alerts the user when the FHRM is not configured properly.

It is another object of the current invention to disclose the FHRM as defined in any of the above, wherein the user guidance alerts the user when a surface is attached to at least one transducer.

It is another object of the current invention to disclose the FHRM as defined in any of the above, wherein the effectiveness of the location of the FHB signal depends on a factor selected from a group consisting of pressure applied on at least one Doppler transducer, speed of movement of at least one Doppler transducer, and any combination thereof.

It is another object of the current invention to disclose the FHRM as defined in any of the above, wherein the communication module alerts when the speed of movement is too high to acquire the FHB signal.

It is another object of the current invention to disclose the FHRM as defined in any of the above, wherein the communication module alerts when the pressure is too low to acquire the FHB signal.

It is another object of the current invention to disclose the FHRM as defined in any of the above, wherein at least one Doppler transducer is operably coupled to an item selected from a group consisting of: (a) a pressure sensing module; (b) an accelerometer; and any combination thereof.

It is another object of the current invention to disclose the FHRM as defined in any of the above, wherein the user guidance alert is selected from a group consisting of: (a) audible; (b) visual; (c) sensible; and any combination thereof.

It is another object of the current invention to disclose the FHRM as defined in any of the above, wherein the location of at least one Doppler transducer in which the FHB signal was detected is stored by at least one processor.

It is another object of the current invention to disclose the FHRM as defined in any of the above, wherein the well-being of the fetus can be assessed by comparing the FHB signal location to the stored location of at least one Doppler transducer in which the FHB signal was previously detected.

It is another object of the current invention to disclose the FHRM as defined in any of the above, wherein change in the position of the fetus can be assessed by comparing the FHB signal location to the stored location of at least one Doppler transducer in which the FHB signal was previously detected.

It is another object of the current invention to disclose the FHRM as defined in any of the above, wherein it can be detected whether the fetus has flipped to a tum-down position by comparing the FHB signal location to the stored location of at least one Doppler transducer in which the FHB signal was previously detected.

It is another object of the current invention to disclose the FHRM as defined in any of the above, wherein it can be detected whether the fetus is under stress by comparing the FHB signal location to the stored location of at least one Doppler transducer in which the FHB signal was previously detected.

It is another object of the current invention to disclose a method for operating a fetal heart rate monitor (FHRM) useful for locating and monitoring fetal heartbeat (FHB) signals, the method comprising steps of: (a) obtaining an FHRM with at least one Doppler transducer, at least one processor and at least one communication module; (b) placing at least one Doppler transducer on the abdomen of an expectant mother; (c) moving at least one Doppler transducer around the abdomen of an expectant mother; and (d) searching for an FHB signal; wherein the method additionally comprises steps of receiving user guidance from the communicating module towards detecting the FHB signal according to feedback of the data received from at least one Doppler transducer and processed by at least one processor.

It is another object of the current invention to disclose the FHRM as defined in any of the above, additionally comprising a step of placing at least one Doppler transducer on the abdomen of a pregnant mother.

It is another object of the current invention to disclose the FHRM as defined in any of the above, additionally comprising a step of generally instructing the user for detecting the FHB signal.

It is another object of the current invention to disclose the FHRM as defined in any of the above, wherein the step of generally instructing includes instructing to start locating the FHB signal at the middle-top part of the abdomen of an expectant mother.

It is another object of the current invention to disclose the FHRM as defined in any of the above, wherein the step of generally instructing is based on previous searches of FHB signals.

It is another object of the current invention to disclose the FHRM as defined in any of the above, wherein additionally comprising a step of adjusting the general instructions s according to general medical knowledge.

It is another object of the current invention to disclose the FHRM as defined in any of the above, wherein additionally comprising a step of adjusting the general instructions according to the week of the gestation.

It is another object of the current invention to disclose the FHRM as defined in any of the above, wherein the step of receiving user guidance from the communicating module includes guidance regarding features selected from a group consisting of: (a) angling; (b) pressure of at least one Doppler transducer on the abdomen of an expectant mother; (c) direction of movement of at least one Doppler transducer on the abdomen of an expectant mother; (d) speed of movement of at least one Doppler transducer on the abdomen of an expectant mother; (e) quality of the FHB signal; and any combination thereof.

It is another object of the current invention to disclose the FHRM as defined in any of the above, additionally comprising a step of selecting the user guidance means from a group consisting of: (a) audible; (b) sensible; (c) visual; and any combination thereof.

It is another object of the current invention to disclose the FHRM as defined in any of the above, wherein the audible guidance are beeping sounds.

It is another object of the current invention to disclose the FHRM as defined in any of the above, wherein the audible guidance are oral instructions.

It is another object of the current invention to disclose the FHRM as defined in any of the above, wherein the visual guidance are flashing lights.

It is another object of the current invention to disclose the FHRM as defined in any of the above, wherein the visual guidance is a graphical map of the abdomen of an expectant mother.

It is another object of the current invention to disclose the FHRM as defined in any of the above, additionally comprising a step of displaying the approximated position of the fetus and the location of the fetus's heart on the graphical map.

It is another object of the current invention to disclose the FHRM as defined in any of the above, additionally comprising a step of basing the sensible guidance on haptic feedback.

It is another object of the current invention to disclose the FHRM as defined in any of the above, additionally comprising a step of selecting the communicating module to be a GUI selected from a group consisting of: (a) laptop; (b) smartphone; (c) computer; (d) tablet; (e) mobile phone; and any combination thereof.

It is another object of the current invention to disclose the FHRM as defined in any of the above, wherein the GUI is a touch screen.

It is another object of the current invention to disclose the FHRM as defined in any of the above, additionally comprising a step of illustrating on the touch screen the graphical map of the abdomen of an expectant mother.

It is another object of the current invention to disclose the FHRM as defined in any of the above, additionally comprising a step of notifying at least one processor through the touch screen on the location of at least one transducer on the abdomen of an expectant mother.

It is another object of the current invention to disclose the FHRM as defined in any of the above, additionally comprising a step of notifying at least one processor through the touch screen on the location of at least one transducer on the abdomen of an expectant mother in which the FHB signal was detected.

It is another object of the current invention to disclose the FHRM as defined in any of the above, wherein additionally comprising a step of controlling the GUI by eye movement.

It is another object of the current invention to disclose the FHRM as defined in any of the above, wherein additionally comprising a step of controlling the GUI by a pointing device.

It is another object of the current invention to disclose the FHRM as defined in any of the above, wherein additionally comprising a step of controlling the GUI by a mouse.

It is another object of the current invention to disclose the FHRM as defined in any of the above, additionally comprising a step of utilizing the location of at least one transducer on the abdomen of an expectant mother in which the FHB signal was detected for future guiding towards the detection of the FHB signal.

It is another object of the current invention to disclose the FHRM as defined in any of the above, additionally comprising a step of uniting at least one processor and the communicating module in one computerized device selected from a group consisting of: (a) smartphone; (b) tablet; (c) laptop; (d) desktop; and any combination thereof.

It is another object of the current invention to disclose the FHRM as defined in any of the above, wherein the step of guiding the user is additionally based on previous searches of FHB signals.

It is another object of the current invention to disclose the FHRM as defined in any of the above, wherein the step of guiding the user is additionally based on the time passed from the previous searches.

It is another object of the current invention to disclose the FHRM as defined in any of the above, wherein the step of guiding the user is additionally based on the position of the fetus.

It is another object of the current invention to disclose the FHRM as defined in any of the above, additionally comprising a step of giving indication when the FHB signal is found.

It is another object of the current invention to disclose the FHRM as defined in any of the above, additionally comprising a step of selecting the indication from a group of indications consisting of: (a) sensible; (b) audible, (c) visual.

It is another object of the current invention to disclose the FHRM as defined in any of the above, additionally comprising a step of instructing the user not to move at least one Doppler transducer when the FHB signal is detected.

It is another object of the current invention to disclose the FHRM as defined in any of the above, additionally comprising a step of alerting the user when the location of the FHB signal is not effective.

It is another object of the current invention to disclose the FHRM as defined in any of the above, additionally comprising a step of alerting the user when the FHRM is not configured properly.

It is another object of the current invention to disclose the FHRM as defined in any of the above, additionally comprising a step of alerting the user when a surface is attached to at least one transducer.

It is another object of the current invention to disclose the FHRM as defined in any of the above, wherein the effectiveness of locating the FHB signal depends on a factor selected from a group consisting of pressure applied on at least one Doppler transducer, speed of movement of at least one Doppler transducer, and any combination thereof.

It is another object of the current invention to disclose the FHRM as defined in any of the above, additionally comprising a step of alerting the user when the speed of movement is too high to acquire the FHB signal.

It is another object of the current invention to disclose the FHRM as defined in any of the above, additionally comprising a step of alerting the user when the pressure is too low to acquire the FHB signal.

It is another object of the current invention to disclose the FHRM as defined in any of the above, additionally comprising a step of operably coupling within at least one Doppler transducer an item selected from a group consisting of: (a) pressure sensing module; (b) accelerometer; and any combination thereof.

It is another object of the current invention to disclose the FHRM as defined in any of the above, additionally comprising a step of selecting the alert from a group consisting of: (a) audible; (b) visual; (c) sensible; and any combination thereof.

It is another object of the current invention to disclose the FHRM as defined in any of the above, additionally comprising a step of storing by at least one processor the location of at least one Doppler transducer in which the FHB signal was detected.

It is another object of the current invention to disclose the FHRM as defined in any of the above, additionally comprising a step of assessing the well-being of the fetus by comparing the FHB signal location to the stored location of at least one Doppler transducer in which the FHB signal was previously detected.

It is another object of the current invention to disclose the FHRM as defined in any of the above, additionally comprising a step of assessing the position of the fetus by comparing the FHB signal location to the stored location of at least one Doppler transducer in which the FHB signal was previously detected.

It is another object of the current invention to disclose the FHRM as defined in any of the above, additionally comprising a step of detecting the fetus has flipped to a tum-down position by comparing the FHB signal location to the stored location of at least one Doppler transducer in which the FHB signal was previously detected.

It is another object of the current invention to disclose the FHRM as defined in any of the above, additionally comprising a step of detecting whether the fetus is under stress by comparing the FHB signal location to the stored location of at least one Doppler transducer in which the FHB signal was previously detected.

It is another object of the current invention to disclose a processor for operating a Doppler transducer useful for locating and monitoring fetal heartbeat (FHB) signals, the processor comprising: (a) an input module for receiving data of the FHB signal generated by the Doppler transducer; (b) an analyzing module for analyzing the data; and (c) an output module for transmitting the analyzed data to a user; wherein the output module comprises user guidance means for guiding the user towards the detection of the FHB signal according to feedback of the data received from at least one Doppler transducer and processed by the processor.

It is another object of the current invention to disclose the FHRM as defined in any of the above, wherein the processor is intended for home use or ambulatory care.

It is another object of the current invention to disclose the FHRM as defined in any of the above, wherein the user guidance means includes general instructions for detection of the FHB signal.

It is another object of the current invention to disclose the FHRM as defined in any of the above, wherein the general instructions include instructions to initiate location of the FHB signal at the middle-top part of the abdomen of an expectant mother.

It is another object of the current invention to disclose the FHRM as defined in any of the above, wherein the general instructions are adjusted by general medical knowledge.

It is another object of the current invention to disclose the FHRM as defined in any of the above, wherein the general instructions are adjusted by the week of gestation.

It is another object of the current invention to disclose the FHRM as defined in any of the above, wherein the user guidance means facilitate user guidance regarding features selected from a group consisting of: (a) angling; (b) pressure of the Doppler transducer on the abdomen of an expectant mother, (c) direction of movement of the Doppler transducer on the abdomen of an expectant mother; (d) speed of movement of the Doppler transducer on the abdomen of an expectant mother; (e) quality of the FHB signal; and any combination thereof.

It is another object of the current invention to disclose the FHRM as defined in any of the above, wherein the user guidance means are selected from a group consisting of: (a) audible; (b) sensible; (c) visual; and any combination thereof.

It is another object of the current invention to disclose the FHRM as defined in any of the above, wherein the audible guidance are beeping sounds.

It is another object of the current invention to disclose the FHRM as defined in any of the above, wherein the audible guidance are verbal instructions.

It is another object of the current invention to disclose the FHRM as defined in any of the above, wherein the visual guidance are flashing lights.

It is another object of the current invention to disclose the FHRM as defined in any of the above, wherein the visual guidance is a graphical map of the abdomen of an expectant mother.

It is another object of the current invention to disclose the FHRM as defined in any of the above, wherein the visual guidance displays location of the Doppler transducer on the graphical map of the abdomen of an expectant mother.

It is another object of the current invention to disclose the FHRM as defined in any of the above, wherein the sensible guidance is based on haptic feedback.

It is another object of the current invention to disclose the FHRM as defined in any of the above, wherein the user notifies the processor through the touch screen with an illustration of the abdomen of an expectant mother on the location of at least one transducer on the abdomen of an expectant mother.

It is another object of the current invention to disclose the FHRM as defined in any of the above, wherein the user notifies the processor through the touch screen with an illustration of the abdomen of an expectant mother on the location of at least one transducer on the abdomen of an expectant mother in which the FHB signal was detected.

It is another object of the current invention to disclose the FHRM as defined in any of the above, wherein the location of at least one transducer on the abdomen of an expectant mother in which the FHB signal was detected can be utilized by the processor for future guiding towards the detection of the FHB signal.

It is another object of the current invention to disclose the FHRM as defined in any of the above, wherein the processor is a GUI selected from a group consisting of: (a) laptop; (b) smartphone; (c) computer; (d) tablet; and any combination thereof.

It is another object of the current invention to disclose the FHRM as defined in any of the above, wherein the processor is a computerized device selected from a group consisting of: (a) smartphone; (b) tablet; (c) laptop; (d) desktop; (e) mobile phone; and any combination thereof.

It is another object of the current invention to disclose the FHRM as defined in any of the above, wherein the user guidance is additionally based on data from previous searches of FHB signals.

It is another object of the current invention to disclose the FHRM as defined in any of the above, wherein the user guidance is based on the previous searches which takes into considerations the time passed from the previous searches.

It is another object of the current invention to disclose the FHRM as defined in any of the above, wherein the user guidance is additionally based on data of the position of the fetus.

It is another object of the current invention to disclose the FHRM as defined in any of the above, wherein the user guidance gives indication when the FHB signal is found.

It is another object of the current invention to disclose the FHRM as defined in any of the above, wherein the indication is selected from a group of indications consisting of: (a) sensible; (b) audible, (c) visual.

It is another object of the current invention to disclose the FHRM as defined in any of the above, wherein the user guidance instructs the user not to move at least one Doppler transducer when the FHB signal is detected.

It is another object of the current invention to disclose the FHRM as defined in any of the above, wherein the user guidance alerts the user when the location of the FHB signal is not effective.

It is another object of the current invention to disclose the FHRM as defined in any of the above, wherein the user guidance alerts the user when the FHRM is not configured properly.

It is another object of the current invention to disclose the FHRM as defined in any of the above, wherein the user guidance alerts the user when a surface is attached to at least one transducer.

It is another object of the current invention to disclose the FHRM as defined in any of the above, wherein the effectiveness of the location of the FHB signal depends on a factor selected from a group consisting of pressure applied on at least one Doppler transducer, speed of movement of at least one Doppler transducer, and any combination thereof.

It is another object of the current invention to disclose the FHRM as defined in any of the above, wherein the communication module alerts when the speed of movement is too high to acquire the FHB signal.

It is another object of the current invention to disclose the FHRM as defined in any of the above, wherein the communication module alerts when the pressure is too low to acquire the FHB signal.

It is another object of the current invention to disclose the FHRM as defined in any of the above, wherein the location of at least one Doppler transducer in which the FHB signal was detected is stored by the processor.

It is another object of the current invention to disclose the FHRM as defined in any of the above, wherein the well-being of the fetus can be assessed by comparing the FHB signal location to the stored location of at least one Doppler transducer in which the FHB signal was previously detected.

It is another object of the current invention to disclose the FHRM as defined in any of the above, wherein change in the position of the fetus can be assessed by comparing the FHB signal location to the stored location of at least one Doppler transducer in which the FHB signal was previously detected.

It is another object of the current invention to disclose the FHRM as defined in any of the above, wherein it can be detected whether the fetus has flipped to a turn-down position by comparing the FHB signal location to the stored location of at least one Doppler transducer in which the FHB signal was previously detected.

It is another object of the current invention to disclose the FHRM as defined in any of the above, wherein it can be detected whether the fetus is under stress by comparing the FHB signal location to the stored location of at least one Doppler transducer in which the FHB signal was previously detected.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured. The present invention provides an FHRM with means to facilitate the search of a heartbeat.

In the accompanying drawings:

FIG. 1 is a block diagram of the FHRM (100);

FIG. 2 shows the beam width of the Doppler transducer in the high and low frequency modes (200);

FIG. 3 shows a two resonance Piezoelectric transducer (300);

FIG. 4 is a schematic flow diagram illustrating the method for operating an FHRM comprising a Doppler transducer with two frequency modes for locating an FHB signal and monitoring the FHR (400);

FIG. 5 is a schematic flow diagram illustrating a method for using one embodiment of the invention in which the Doppler transducer has two frequency modes with an automatic switch and system's guidance (500);

FIG. 6 is a schematic flow diagram illustrating a method for using one embodiment of the invention in which the Doppler transducer has two frequency modes with an automatic switch, system's guidance and a Multi Toggle (600);

FIG. 7 is a schematic flow diagram illustrating a method for using one embodiment of the invention in which the Doppler transducer has two frequency modes with an automatic switch and graphical guidance (700);

FIG. 8 is a schematic flow diagram illustrating the method for operating an FHRM comprising a Doppler transducer with two intensity modes for locating an FHB signal and monitoring FHR (800);

FIG. 9 is a schematic flow diagram illustrating a method for using one embodiment of the invention in which the Doppler transducer has several beam intensity modes with an automatic switch and system's guidance (900);

FIG. 10 is a schematic flow diagram illustrating a method for using one embodiment of the invention in which the Doppler transducer has several beam intensity modes with an automatic switch, system's guidance and sensor locator (1000);

FIG. 11 is a schematic flow diagram illustrating the method for operating an FHRM comprising a Doppler transducer operably coupled to a pressure sensing module, for locating an FHB signal and monitoring the FHR (1100);

FIG. 12 is a schematic flow diagram illustrating a method for using one embodiment of the invention in which the Piezoelectric ceramic element of the Doppler transducer is additionally used as a pressure sensing module (1200);

FIG. 13A is a schematic diagram of a bottom view of a Doppler transducer with a single Piezoelectric ceramic element functioning also as a pressure sensor (1300);

FIG. 13B is a schematic diagram of a side view of a Doppler transducer with a single Piezoelectric ceramic element functioning also as a pressure sensor (1300);

FIG. 14A is a schematic diagram of a bottom view of a Doppler transducer with a plurality of Piezoelectric ceramic elements functioning as pressure sensors (1400).

FIG. 14B is a schematic diagram of a side view of a Doppler transducer with a plurality of Piezoelectric ceramic elements functioning as pressure sensors in which equal pressure is applied on all Piezoelectric ceramic elements (1400).

FIG. 14C is a schematic diagram of a side view of a Doppler transducer with a plurality of Piezoelectric ceramic elements functioning as pressure sensors in which different amounts of pressure are applied by the abdomen of a pregnant woman (1400).

FIG. 15 is a schematic flow diagram illustrating the method for operating an FHRM comprising user guidance means for guiding said user towards the detection of said FHB signal according to said processed data. (1500); and

FIG. 16 is a schematic flow diagram illustrating one embodiment of the invention (1600); and

FIG. 17 is an illustration of a Doppler transducer positioned on the abdomen of a pregnant woman and a graphical map of the same abdomen on a GUI showing the position of the Doppler transducer (1700).

### DETAILED DESCRIPTION OF THE INVENTION

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The essence of the present invention is to provide a fetal heart rate monitor (FHRM) with a ultrasound Doppler transducer using high and low frequency modes as well as high and low intensity modes for facilitating the FHB signal localization search, for receiving accurate readings and decreasing the amount of energy transmitted to the body of the expectant mother and the fetus and for optimizing the device's energy consumption. The essence of the present invention is to further provide an FHRM with a pressure sensor to detect the pressure of the abdomen on the Doppler transducer. The present invention additionally provides an FHRM with use guidance.

A Doppler monitor with an easy location of signal will increase the safety of the device as shorter sessions will be needed which will reduce the amount of energy transduced to the Mother and fetus. In addition, this kind of monitor will assure the mother of the well-being of the fetus which prevents stress reactions as well as unnecessary doctor appointments.

As will be shown, the present invention provides a practical means and method for enabling an untrained person, e.g. an expectant mother, to monitor accurately, easily the FHB. An important stage in this process is to be able to locate the FHB signal. None of the prior art technologies deal with improving the location procedure of the FHB signal for an untrained person.

The term “fetal heart rate monitor (FHRM)” refers hereinafter to a device used to detect the heartbeat of a fetus for prenatal care. It is comprised of at least a Doppler transducer, a wave generating module, a communicating module, a processor and other auxiliary electronics.

The term “Doppler transducer” refers hereinafter to the module in the FHRM that utilizes the Doppler effect to convert the movement of the fetus's heart into sound waves.

The term “FHB signal location” refers hereinafter to locating the optimal position for placing the Doppler transducer on the mother's abdomen, in order to facilitate an accurate reading of the FHR from the Ultrasound signal reflected from the fetus' heart valves. Several filtering and processing techniques exist which enables the identification of the FHB signal. For example, patent application EP2346408 discloses filters to reject signals from stationary and slowly moving tissue. Another example is patent application US20130123637 which removes sidebands and performs an anti-aliasing filtering.

The term “plurality” refers hereinafter to two or more units of an object.

The term “Piezoelectric ceramic element” refers hereinafter to an element that uses the Piezoelectric effect to measure pressure, acceleration, strain or force by converting them to an electrical charge. The Piezoelectric ceramic element is a transceiver of ultrasonic waves by its microscopic vibrations which are useful for detecting FHB signals by measuring the change in the frequency of the reflecting waves. The Piezoelectric ceramic element is also useful for measuring pressure as its electric capacity increases when pressure increases.

The term “pressure sensing module” refers hereinafter to a module that generates a signal as a function of the pressure imposed. For example, the pressure sensing module can be an absolute pressure sensor, gauge pressure sensor, vacuum pressure sensor, differential pressure sensor, sealed pressure sensor. In some of the embodiments the pressure sensor can be the Piezoelectric ceramic element used for transceiving ultrasonic waves for detecting the FHB signal. The Piezoelectric ceramic element detects pressure by changes in its electric capacity as a result of said pressure; the electric capacity increases as the pressure increases.

The term “angling” refers hereinafter to spherical rotation. More specifically, the term relates to the rotation of the Doppler transducer to change the directionality of the Piezoelectric ceramic element.

The term “center of pressure point” refers hereinafter to the point of application of the ground reaction force vector. The ground reaction force vector represents the sum of all forces acting between a physical object and its supporting surface.

The term “properly configured” refers hereinafter to a device that is configured in a way that enables it usage. More specifically the term refers to configuring a fetal heart rate monitor that enables acquiring a heartbeat signal if such a signal exists.

The term “user guidance means” refers hereinafter to a set of instructions used to direct the user of an FHRM towards the location of an FHB signal. The instructions include direction of movement of the Doppler transducer, direction of angling of the transducer, speed of movement of the transducer, when to stop moving the transducer, and when to apply stress to the transducer. The instructions are determined according to feedback from the transducer (the signal it acquires). The instructions may also be affected from general medical data (averaged e.g. data of FHB search), personal medical data (e.g. week of gestation, position of the fetus), and data from previous FHB searches.

The term “haptic feedback” refers hereinafter to a technology which takes advantage of the sense of touch by applying forces, vibrations, or motions to the user. This mechanical stimulation can be used to assist in the creation of virtual objects in a computer simulation, to control such virtual objects, and to enhance the remote control of machines and devices

The term “general instructions” refers hereinafter to any instructions given on the basis of general medical knowledge and does not involve specific data concerning a specific patient. This general knowledge is based on averaging a vast amount of medical cases. More specifically the term relates to instructions for locating an FHB signal based on general data regarding the pregnancy at that stage. For instance, the location on the abdomen in which it is most likely to find the FHB signal at that stage of gestation and the average amount of pressure in which the FHB signal is usually detected.

The term “general medical knowledge” refers hereinafter to any knowledge based on medical research, statistical data and clinical experience. More specifically, the term refers to any information regarding the fetal heart rate which is extracted from general medical knowledge.

The term “flipping” refers hereinafter to the final flip of the fetus in the uterus into a head down position ready for labor. Once the uterus has flipped, the fetus stays in the head down position until labor and is not able to flip back again.

Reference is now made to FIG. 1, which shows a block diagram of the FHRM. The FHRM is comprised of a Doppler transducer (101) that has either two frequency modes, two intensity modes or two frequency modes combined with two intensity modes. The transducer is connected to a processor (102) that is responsible for identifying the signal and converting it into a reading, switching between modes according to signals and transmitting data to the communicating module (103). The communicating module passes all information from the processor to the user which includes guidance to find the heartbeat signal and the heartbeat signals as well as additional information like analysis of the signal. A computerized device can include both the processor and the communicating module. For example, a smartphone or a tablet can be connected to transducer and their processor will function as the monitor's processor and their GUI as the communicating module.

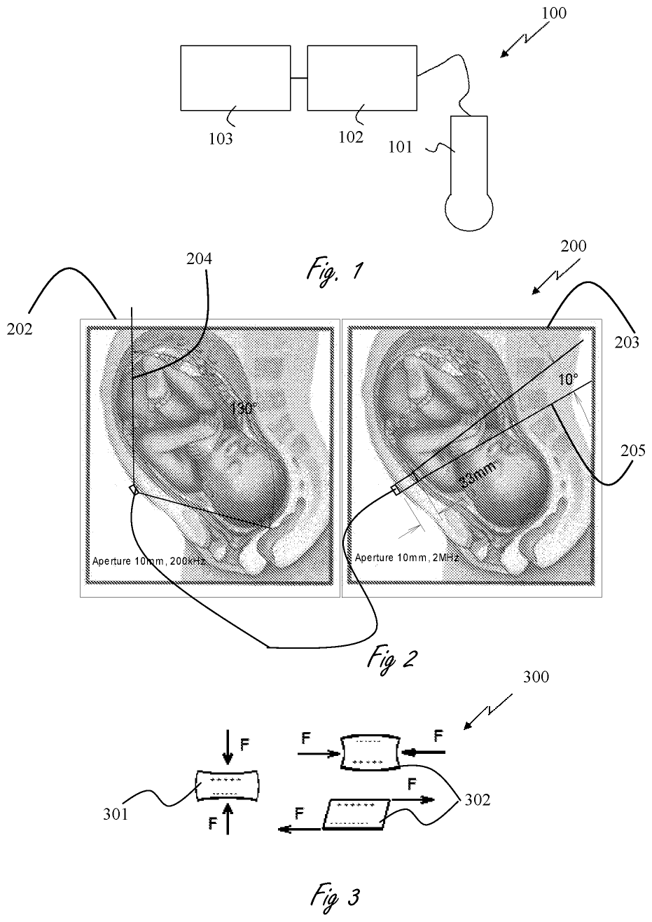

Reference is now made to FIG. 2, which shows the beam width in the high and low frequency modes (200) of the transducer (201). The low frequency (202) mode allows simple detection as the beam width is relatively wide (204). For example, if the frequency is 200 kHz than the beam angle is about 130° producing a wide beam. A wide beam simplifies the search since it will detect the FHB in a relatively wide range of locations as it covers more of the abdomen. This will enable an untrained person to detect the FHB relatively easily. However, the signal received with the low frequency beam might be of relatively poor quality and insufficient to receive accurate data regarding the FHR as the downside of the wide beam is more noise resulting in a signal extraction problem. Therefore, once the FHB is detected in the low frequency mode the transducer automatically or manually changes to the high frequency mode (203) which has a narrow beam (205) that has an angle of about 10° that provides a higher quality signal. The combination of high and low frequencies in one transducer allows simple location of the FHB signal without compromising on the signal quality.

Reference is now made to FIG. 3, which shows a Piezoelectric transducer with two resonance frequencies (300). A radial low frequency wide beam (−200 KHz) (301) used for preliminary search and location of the FHB and a natural high frequency narrow beam (−2 MHz)(302) used for focused and accurate FHR reading.

More than two frequencies might ease the search process even more as it will enable gradual increase of the intensity while in parallel the beam narrows and focuses on the fetal heart. In this method the location of the beam begins with the lowest frequency searching all around the abdomen. Once an FHB signal is detected there is still a wide range of movement for improving the signal so the frequency increases by a bit while the beam narrows. In this new mode that signal may not be detected any more so either the transducer moves slowly around the area where the FHB signal was detected or the beam can return to the high frequency. After several iterations as described, the ideal location of the transducer will be found on the abdomen of the expectant mother.

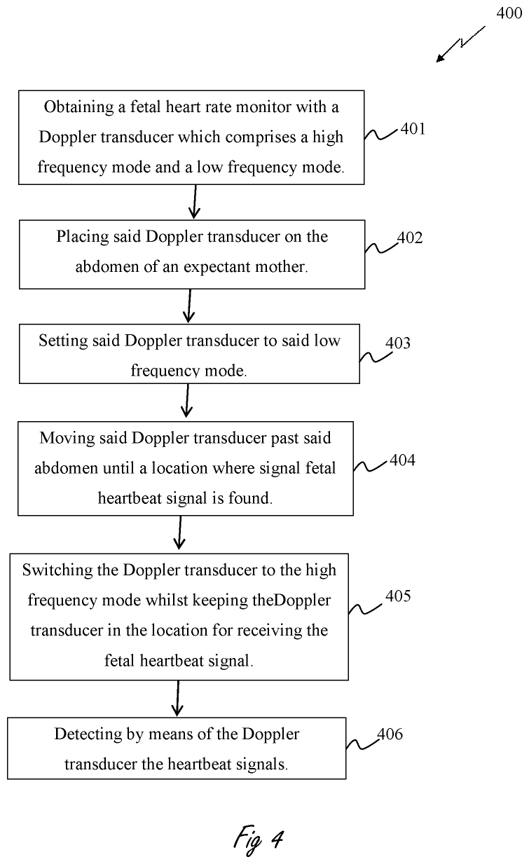

Reference is now made to FIG. 4, which is a schematic flow diagram illustrating the method for operating an FHRM for locating and monitoring an FHB signal (400). In the first step an FHRM with a Doppler transducer is obtained (401). The transducer has at least two frequency modes, high and low. In the second step the Doppler transducer is placed on the abdomen of an expectant mother (402). The user of the monitor can be the expectant mother itself, a medical staff member (physician, technician) or any other person like a family member or a friend. In the next step the transducer is set to the low frequency mode (403). The frequency mode can be automatically set to this mode when turning on the transducer or should be set manually by the user. Once the transducer is set to the low frequency mode the transducer is moved around the abdomen of the expectant mother for locating the FHB signal (404). When the signal is found the transducer switches to a high frequency mode (405) either manually or automatically. Automated switching to the high frequency occurs once a signal sufficient to sustain accurate measurement of FHR is recognized. In this case the threshold of the signal is to be determined to cause the frequency switching. Numerous filtering and processing methods can be applied in order to obtain an accurate signal. In the last step, the FHB signal is detected (406) in high quality with the high frequency beam. Signals indicative of the FHR are collected until sufficient for the user and then the transducer can be turned off If during signal collection the signal disappears it can be relocated again by switching the transducer back to the low frequency mode and searching along the abdomen.

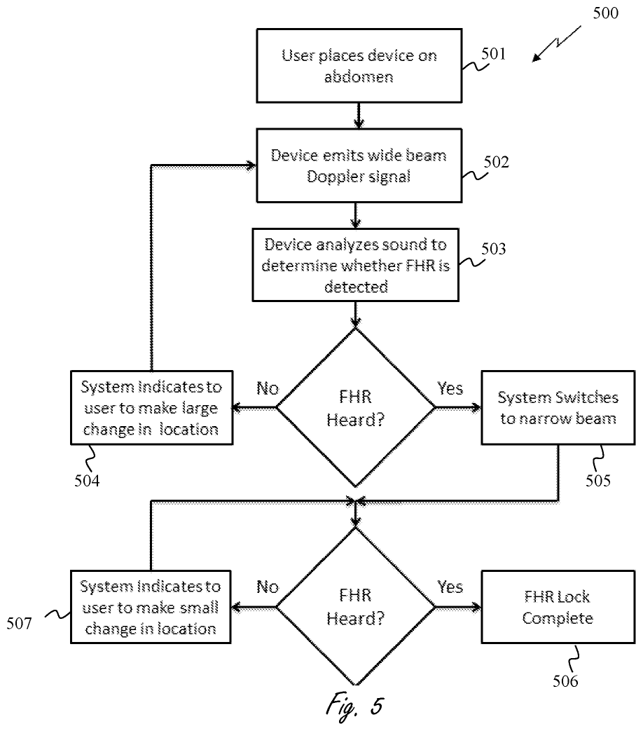

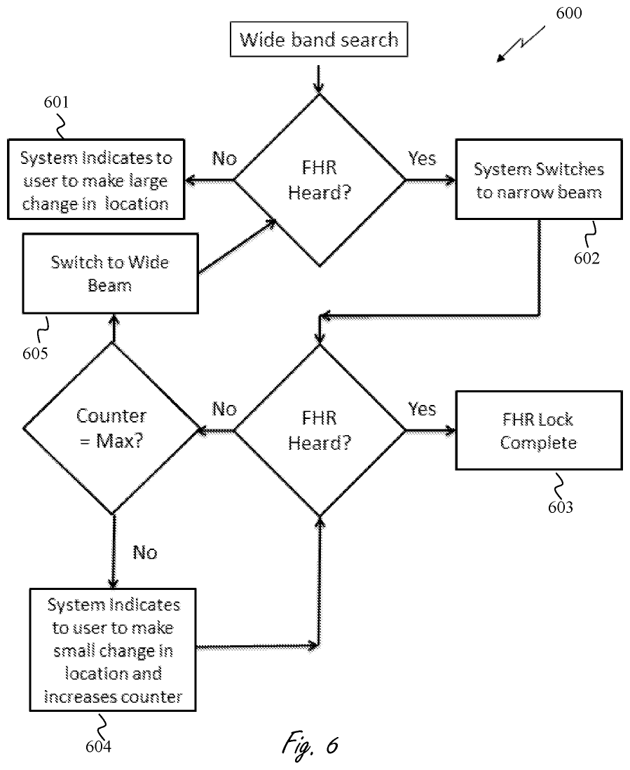

Reference is now made to FIG. 5, which is a schematic flow diagram illustrating one embodiment of the invention. In this embodiment, the low and high frequency modes are implemented together in an FHRM with user guidance. The user places the Doppler transducer, set to the low frequency mode, on the abdomen of an expectant mother (501). The user moves the transducer around the abdomen (502) while the processor of the FHRM determines whether an FHB signal is sufficient to sustain accurate measurement of FHR (503). If a signal is not detected, then the system indicates the user to make large changes in location of the transducer (504). If a signal is detected, then the system switches to the high frequency mode having the narrow beam (505) and the processor searches again for an FHB signal. If the signal is heard, then the transducer locks (506) and FHR can be detected. If by switching to the high frequency mode the FHB signal is lost, the system indicates to the user, by light or sound, to make small changes in the location of the transducer (507). If the small changes do not enable the location of the FHB signal, then the system switches back to the low frequency mode (506).

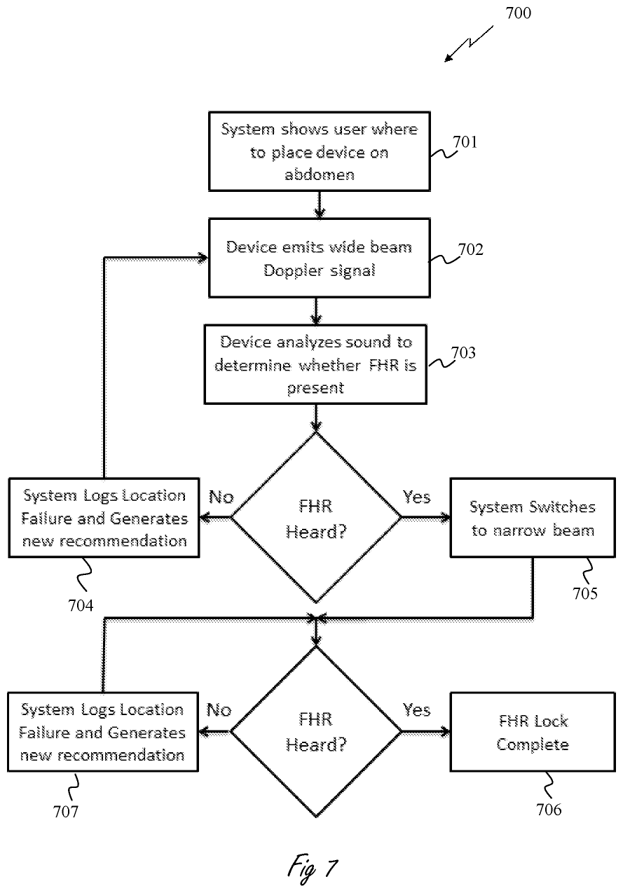

Reference is now made to FIG. 6, which is a schematic flow diagram illustrating another embodiment of the invention. In this embodiment the low and high frequency modes are implemented together in an FHRM with user guidance and system's feedback. Similar to the embodiment in FIG. 5, the user places the Doppler transducer, which is set to the low frequency mode, on the abdomen of an expectant mother and moves it around the abdomen. Each time the transducer stops, the processor of the FHRM determines whether an FHB signal sufficient to sustain accurate measurement of FHR is detected. If signal is not detected, the system indicates, by light or sound, to the user to make large change in location of the transducer (601). If the FHB signal is detected, then the system switches to the high frequency mode having the narrow beam (602) and the processor searches again for an FHB signal. If the signal is heard than the transducer locks (603) and FHR can be detected. If by switching to the high frequency mode the FHB signal is lost, the system indicates to the user, by light or sound, to make small changes in the location of the transducer (604). The system then counts a certain number of attempts to find the FHB signal in the high frequency mode, and if that number is reached then the system switches back to the low frequency mode (605) and the search for signal begins from the beginning. The attempts can be counted according to length of the time that passes since switching to the high frequency mode or by counting the number of attempts of the system to receive an FHB signal.

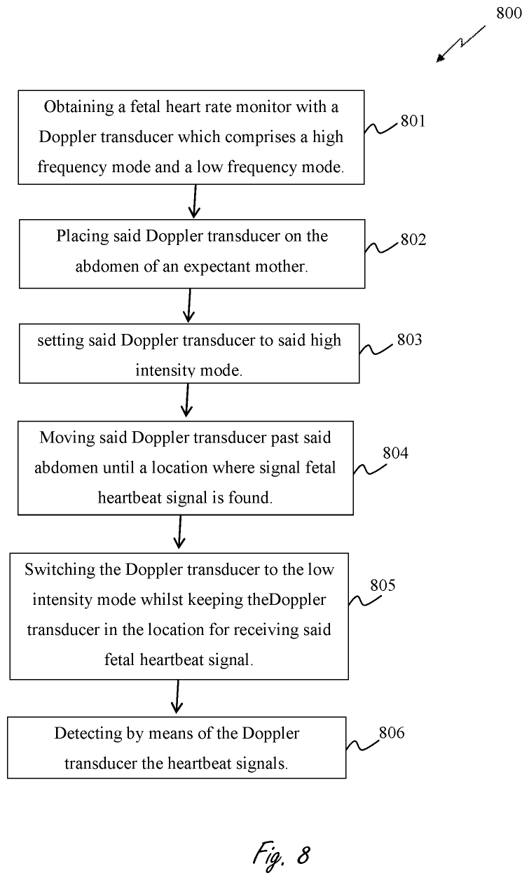

Reference is now made to FIG. 7, which is a schematic flow diagram illustrating another embodiment of the invention. In this embodiment, the low and high frequency modes are implemented together in an FHRM with graphical user guidance. In the first step, the system shows the user on a graphical map of the abdomen where to place the Doppler transducer on the abdomen of the expectant mother (701). The system guides the user where to place the transducer according to data regarding locations with high probability to find in them the FHB signal. These locations can be predicted according to general information regarding the stage of the gestation and according to the location of FHB signal in previous searches. The transducer emits in the low frequency mode resulting in a wide beam (702) and the monitor then analyzes whether a signal is detected (703). If no FHB signal is detected, the system generates a new recommendation for locating the transducer (704). If a signal is detected, the system switches to the high frequency mode having the narrow beam (705). If the FHB signal is detected in the high frequency mode, the system locks and FHR is monitored (706). If the FHB signal is no longer detected, the system generates a recommendation of re-localizing the transducer (707). The system keeps giving new recommendations until the FHB signal is detected in the high frequency mode.

Reference is now made to FIG. 8, which is a schematic flow diagram illustrating the method for operating a Doppler transducer for locating FHB signal and monitoring FHR (800). In the first step, an FHRM with a Doppler transducer is obtained (801). The transducer has at least two modes of high intensity and low intensity. In the second step, the Doppler transducer is placed on the abdomen of an expectant mother (802). The user of the monitor can be the mother itself, a medical staff member (physician, technician) or any other person like a family member or a friend. In the next step, the transducer is set to the high intensity mode (803). The high intensity mode has high sensitivity, and therefore the search for the FHB signal will be easier in this mode. The disadvantages of the high intensity mode are that it transmits high amount of energy into the expectant mother's body that may exceed the regulatory recommendations as well as it consumes a lot of energy that shortens battery life of the FHRM. The intensity mode can be automatically set to this mode when turning on the FHRM or should be set manually by the user. Once the transducer is set on the high intensity mode, the transducer is moved around the abdomen of the expectant mother for locating the FHB signal (804). When the signal is sufficient to sustain accurate measurement of FHR is found, the transducer switches to a low intensity mode (805) either manually or automatically. The low intensity mode reduces the amount of energy transmitted to the expectant mother to not exceed the regulatory recommendations and in addition it saves battery. The sensitivity of the low intensity mode is decreased in comparison to the sensitivity of the high intensity mode but will still create a high-quality signal since the best location on the abdomen was found (the second factor that affects the signal quality). In the last step, the FHB signal is detected (806) in high quality with the low intensity beam. Signals are collected until sufficient for the user, and the transducer can be turned off If during signal collection it disappears, FHB signal can be relocated by switching the high intensity mode back and searching along the abdomen.

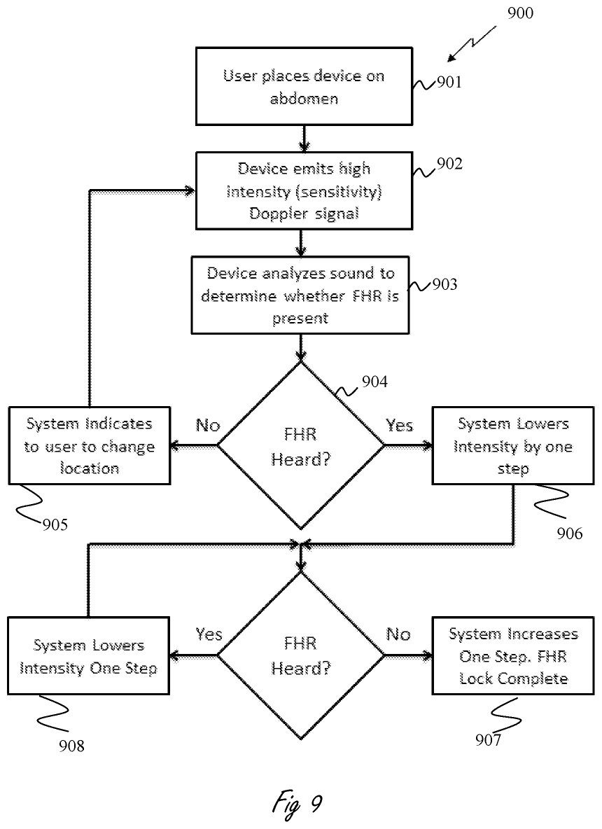

Reference is now made to FIG. 9, which is a schematic flow diagram illustrating another embodiment of the invention. In this embodiment, a Doppler transducer with several intensity modes are implemented together in an FHRM with user guidance. The user places, the Doppler transducer (901), set to the highest intensity mode (902), on the abdomen of an expectant mother. The user moves the transducer around the abdomen, and the processor of the FHRM determines whether an FHB signal is detected (905). If no FHB signal is detected, then the system indicates the user to make large changes in location (905). If an FHB signal is detected, then the system lowers the intensity by one step (906) and the processor searches again for an FHB signal (907). If the signal is detected, then the transducer reduces the beam intensity an additional step (908). Steps 906 and 907 are repeated until a signal is found in the lowest possible beam intensity and FHR can be detected. If by switching to the low intensity mode the FHB signal is lost, the system increase the beam intensity by one step (907) until the FHB signal is detected again.

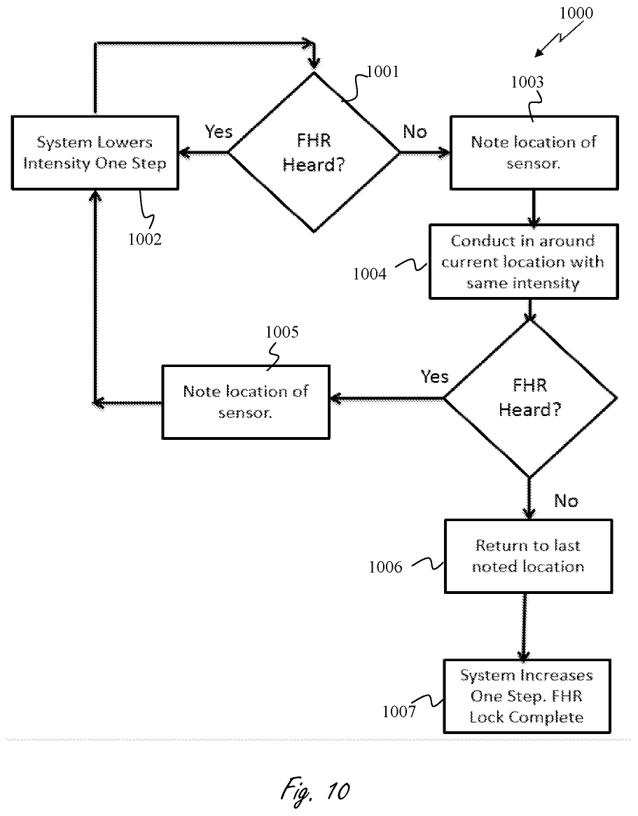

Reference is now made to FIG. 10, which is a schematic flow diagram illustrating another embodiment of the invention. In this embodiment, a Doppler transducer with several intensity modes is implemented in an FHRM which is able to find the point where minimal beam intensity is needed to detect an FHB signal and set minimal intensity. Similar to the previous embodiment, the user places the Doppler transducer, set to the highest intensity mode, on the abdomen of an expectant mother. The user moves the transducer around the abdomen, and the processor of the FHRM determines whether an FHB signal is detected (1001). If an FHB signal is detected, then the system lowers the intensity by one step (1002) and re-tests to determine whether the FHB is detected at the lower intensity (1001). If no signal is detected, the processor notes the location of the transducer (1003) and directs the user to conduct it to a different location while maintaining the lower intensity (1004). While searching for a new location, if an FHB signal is detected, then the processor notes the location and the system lowers the intensity by one step (1005). If the FHB signal is not detected after a given period of time, or if the user indicated he would like to conclude the search, the system directs the user to return back to the last noted location (1006) where the FHB was located successfully. The intensity is raised one step (1007) in order to facilitate FHB reading and the search is concluded. Optionally, the system may test for FHB one more time to assure that the transducer was returned to the proper position and FHB reading can be facilitated.

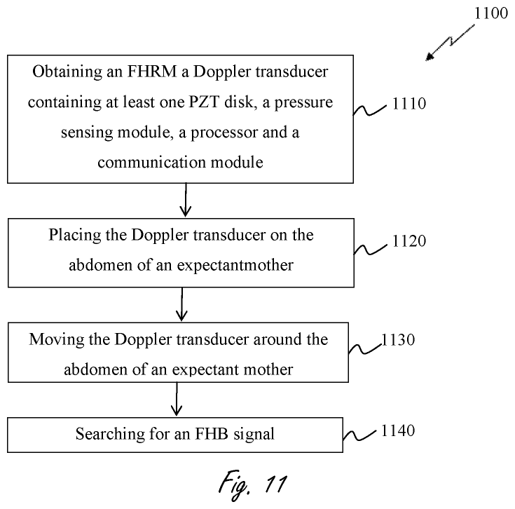

Reference is now made to FIG. 11, which is a schematic flow diagram illustrating the method for operating an FHRM comprising a Doppler transducer operably coupled to a pressure sensing module, for locating an FHB signal and monitoring the FHR (1100). In the first step, an FHRM is obtained (1110) that has a Doppler transducer containing at least one Piezoelectric ceramic element. The FHRM is further comprised of an operably coupled pressure sensing module, a processor and a communication module. It is most likely that the pressure sensing module is located on the surface of the transducer that is in touch with the abdomen of the expectant mother. The pressure sensing module can contain only one pressure sensor or a plurality of them if the directionality of the pressure is also of interest. The Doppler transducer is placed on the abdomen of an expectant mother (1120) in order to try to detect an FHB signal. In the next step, the Doppler transducer is moved around the abdomen of the mother (1130) and the FHB signal is searched (1140). In this method, the pressure sensing module can indicate whether enough pressure is applied to the transducer necessary for acquiring the FHB signal. There is an option for the communication module to alert when not enough pressure is applied. A different option is that the processor turns off the transducer when not enough pressure is applied and only turns it on when sufficient pressure is sensed. If there is a plurality of pressure sensors, then the directionality of the pressure can also be calculated. In this case, the communicating module can direct the user at what angle of the transducer the FHB signal is detected. It can also direct the user to move in a direction opposite to the directionality of the pressure in order to improve the signal.

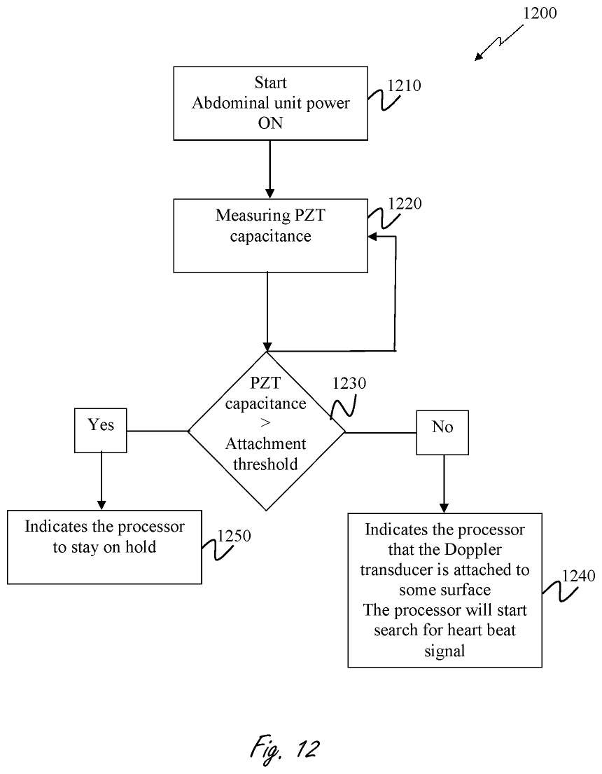

Reference is now made to FIG. 12, which is a schematic flow diagram illustrating a method for using one embodiment of the invention in which the Piezoelectric ceramic element of the Doppler transducer is additionally used as the pressure sensing module (1200). In the first step, the power is turned on (1210) followed by measuring the electric capacity of the Piezoelectric ceramic elements in the Doppler transducer, the measurements done without transmission of US energy (1220). The lower the electric capacity is, the higher the pressure is. If the pressure exceeds a certain threshold, meaning that the PZT electric capacity is below a certain level (1230), then the processor indicates that the Doppler transducer is attached to a surface, it will tum the US transmission for Doppler signal acquisition on and start searching for an FHB signal (1240). If the pressure does not exceed a certain threshold, the processor indicates the transducer to stay on hold until enough pressure is detected (1250) and only then it starts searching for a signal. The pressure indication can be continuous or in a time resolved manner during the time of the FHR monitoring—once not enough pressure is applied the transducer stops the FHB signal search.

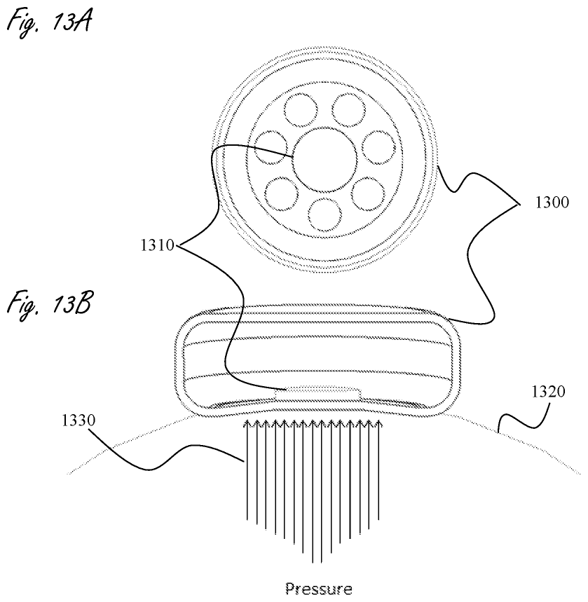

Reference is now made to FIG. 13, which is a schematic diagram of a Doppler transducer with a single Piezoelectric ceramic element functioning also as a pressure sensor (1300). FIG. 13A is a bottom view of the transducer (1300). and the single Piezoelectric ceramic element serving also as a pressure sensor.

FIG. 13B is a side section of the same Doppler transducer placed on an abdomen of a pregnant woman (1320). In the described embodiment, the Doppler transducer serves as a pressure sensor which can sense the pressure of the abdomen of a pregnant woman against the transducer (1330). If sufficient pressure is applied for detection of the FHB signal and for acquiring accurate fetal heart rate, then the transducer starts searching for the signal. If not enough pressure is applied, then the transducer stops transmitting Ultrasound waves. It is also possible that the system alerts when there is not enough pressure by beeping sounds or flashing lights or sends suitable indication via the communication module to the user interface module. In a preferred embodiment, both options can be combined.

This feature serves as troubleshooting guidance—if a user fails to acquire a signal, he/she can know if the device is functioning and only insufficient pressure is applied or that there is another problem. This may prevent stressful situations caused by devices for home use in which an expectant mother is not able to acquire an FHB signal when the problem is only insufficient pressure. The embodiment will additionally assist in reducing the amount of radiation transmitted to the body and enhance energy efficiency and power consumption, as it will transmit ultrasound waves only when there is enough pressure that enables receiving a high quality signal. This will prevent futile measurements that will not lead to signal acquiring and will only lead to unnecessary exposure to ultrasound radiation and waste of battery life

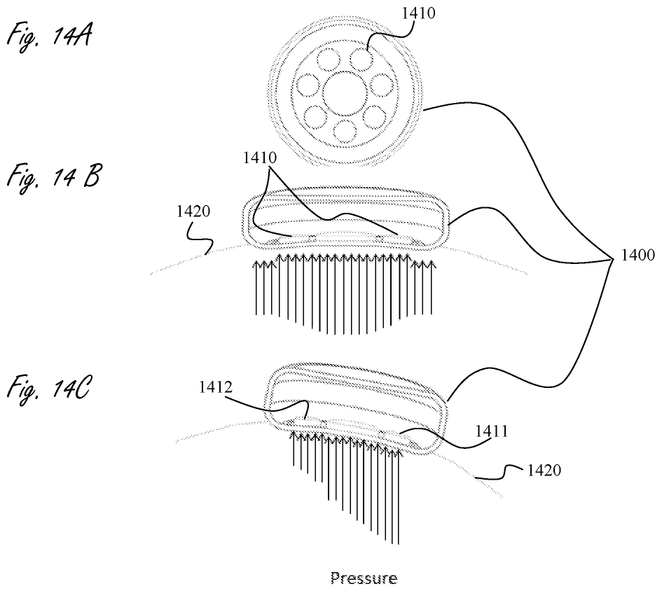

Reference is now made to FIG. 14, which is a schematic diagram of a Doppler transducer with a plurality Piezoelectric ceramic element functioning also as pressure sensors (1400). In this embodiment, a plurality of Piezoelectric ceramic elements are arranged around one central Piezoelectric ceramic element in a Doppler transducer (1410). However, the Piezoelectric ceramic elements can be arranged in various other manners on the surface of the transducer. The plurality of Piezoelectric ceramic elements serves as pressure sensors. They may also have a role in acquiring the FHB heart beat signal. Since there is a plurality of pressure sensors, they have the ability to detect pressure as well as to determine the directionality of that pressure. A bottom view of this embodiment can be seen FIG. 14A.

FIG. 14B is a side view of the Doppler transducer (1400) in which equal pressure is applied on all Piezoelectric ceramic elements (1430) by the abdomen of a pregnant woman (1420). In this case, there is no directionality to the pressure.

FIG. 14C is a side view of the Doppler transducer (1400) in which different amounts of pressure (1430) are applied by the abdomen of a pregnant woman (1420). One Piezoelectric ceramic element (1411) receives more pressure than the other (1412). The different pressures are a result of angling of the transducer, meaning trying to receive a signal by tilting the device in different angles without moving it around the abdomen. The angling is carried out in a direction opposite to the directionality of the pressure. If by angling a better signal is acquired, it is recommended to move the transducer in a direction opposite to the pressure directionality. It is most likely that movement of the transducer in that direction and placing it on the abdomen with no angling will produce an improved signal. In one of the preferred embodiments, the FHRM directs the user to move the transducer in a direction opposite to the pressure directionality sensed by the plurality of Piezoelectric ceramic elements in which a signal was acquired. This is a technique for guiding the user how to improve the signal to receive better FHR reads. The guiding can be audible or visual or it may involve a GUI with a map of the abdomen showing the user exactly where to move the transducer.

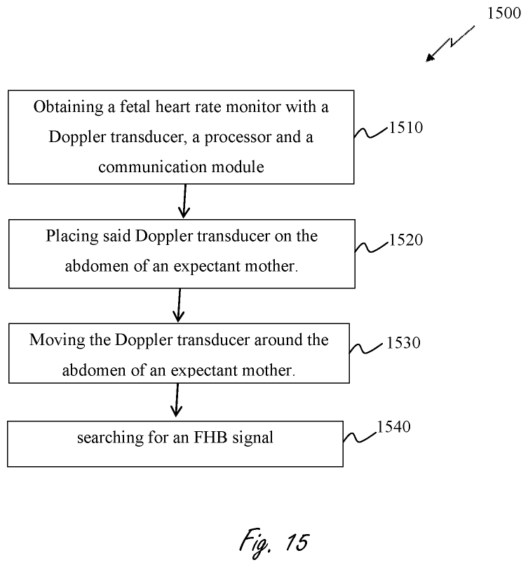

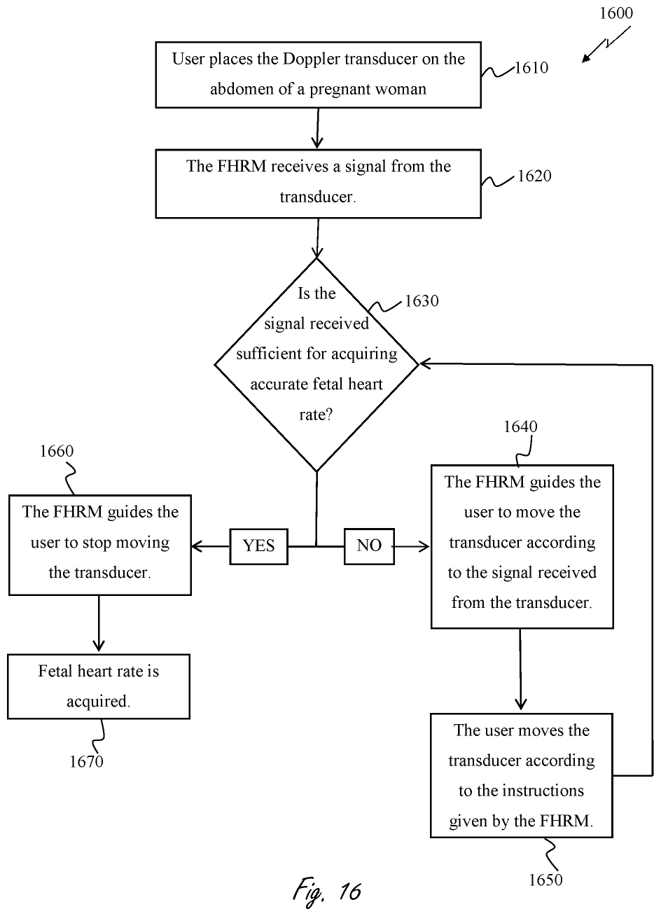

Reference is now made to FIG. 15, which is a schematic flow diagram illustrating the method for operating an FHRM comprising user guidance means for guiding said user towards the detection of said FHB signal according to said processed data. (1500). In the first step, an FHRM is obtained comprising a Doppler transducer, a processor and a communication module (1510). The communication module provides user guidance to facilitate the FHB signal detection. The guidance is primarily based on the processed data received from the Doppler transducer but it may also be based on general medical knowledge and data received and saved of previous searches (e.g., the week of the gestation, general medical knowledge, the location of the FHB in the last search, have the fetus already flipped). In the second step, the Doppler transducer is placed on the abdomen of an expectant mother (1520). The communicating module may guide the user where to place the transducer to ideally start the FHB signal search (e.g. to begin the search at the middle-top part of the abdomen). The user then moves the transducer around the abdomen of the expectant mother (1530) according to the guidance of the communicating module searching for an FHB signal (1540). The guidance can be regarding the direction of movement of the transducer, angling, speed of movement and when a signal is acquired and the transducer should not be moved.

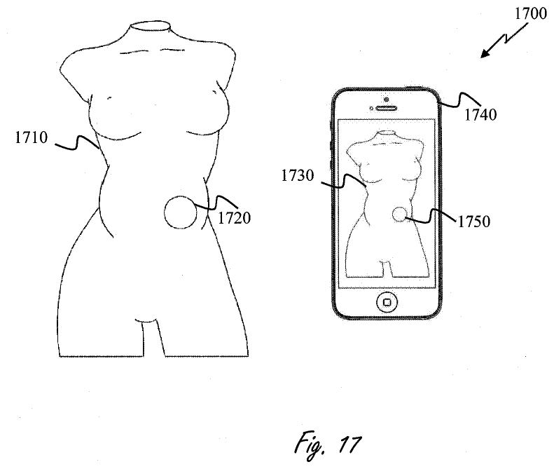

Reference is now made to FIG. 16, which is a schematic flow diagram illustrating one embodiment of the invention (1600). The process begins by placing the Doppler transducer of the FHRM on the abdomen of a pregnant woman (1610). The location for placing the transducer may be random or suggested by the guiding means of the FHRM (e.g. the middle upper part of the abdomen). Once the transducer is placed on the abdomen, it starts receiving signals (1620) which are processed by the processor of the FHRM. The processor determines whether the signal is sufficient for acquiring accurate fetal heart rate (1630) and directs the user accordingly. If the signal is not sufficient, the system guides the user to move the transducer (1640). It may also guide the user regarding angling and amount of pressure to be applied on the transducer. The guidance is majorly based on the signal, but it may also be based on other feedback. For example, it may be based on data from previous searches, general relevant medical data, data regarding the position of the fetus and the week of the gestation. The system may also be a learning system. The user moves the transducer according to the guidance of the system (1650). The guidance can be audible, for example, verbal guiding, or visual in the form of flashing arrows on the transducer showing where to move it. The visual guidance can also be on a GUI presenting a map of the abdomen showing the location of the transducer and directions where to move it. If the signal is sufficient for receiving accurate fetal heart rate, the system guides the user to stop moving the transduce (1660) and fetal heart rate can be acquired (1670).

Reference is now made to FIG. 17, which is an illustration of a Doppler transducer positioned on the abdomen of a pregnant woman and a graphical map of the same abdomen on a GUI showing the position of the Doppler transducer (1700). In one embodiment, the user may notify the processor through a GUI on the location of the Doppler transducer (1710) on the abdomen of a pregnant mother (1720) by showing on a graphical map of the abdomen (1730) on a GUI (1740) (a smart phone in the illustration) the location of the transducer. The user can show the location by a touch screen, through eye movements or by pointing out with a mouse or any other device specified for this use. In another embodiment of the invention, the processor shows the user on the GUI (1740) the location of the transducer on the abdomen of the pregnant mother (1720). In both embodiments, it is possible to save the locations, and especially those in which a heartbeat was detected. Saving these locations may help the user to locate the heartbeat in a future search or the processor can utilize these locations for guiding the user in forthcoming searches.

1. A fetal heart rate monitor useful for locating and monitoring fetal heartbeat signal, said monitor comprising:
at least one Doppler transducer for acquiring said fetal heartbeat signal;
at least one processor configured for processing data received from said at least one Doppler transducer and for determining whether the data is sufficient for acquiring and monitoring the fetal heartbeat signal and, if not sufficient, for providing guidance to a user to make location changes; and,
at least one communication module for providing the guidance to the user; wherein said guidance comprises guiding instructions to detect said fetal heartbeat signal according to feedback received from said at least one Doppler transducer and processed by said at least one processor,
wherein the guidance comprises verbal instructions comprising directions where to move the Doppler transducer on an abdomen of an expected mother.

2. The monitor of claim 1, wherein said guidance include verbal instructions for placing the Doppler transducer at the middle top part of the abdomen of the expected mother.

3. The monitor of claim 1, wherein said guidance relates to an expected position of a fetus according to a week of gestation of the expectant mother.

4. The monitor of claim 1, wherein said guidance comprises features selected from a group consisting of: (a) angling; (b) pressure of said at least one Doppler transducer on an abdomen of the expectant mother; (c) quality of said fetal heartbeat signal; and any combination thereof.

5. The monitor of claim 1, wherein said guidance comprises user guidance that are selected from a group of consisting of: (a) audible guidance; (b) haptic guidance; (c) visual guidance; and any combination thereof.

6. The monitor of claim 1, wherein said verbal instructions further comprises a speed of movement of the at least one Doppler transducer.

7. The monitor of claim 1, wherein said guidance further comprises visual guidance selected from at least one of: a graphical map of an abdomen of an expectant mother and flashing lights.

8. The monitor of claim 1, wherein said graphical map displays an approximate position of the fetus and the location of said fetus's heart.

9. The monitor of claim 1, wherein said graphical map displays to said user the location of said at least one Doppler transducer on said graphical map of said abdomen of the expectant mother.

10. The monitor of claim 1, wherein said at least one communicating module is a graphical user interface (GUI) included in a device selected from a group consisting of: (a) laptop; (b) smartphone; (c) computer; (d) tablet and (e) mobile phone.

11. The monitor of claim 10, wherein said at least one communicating module includes a touch screen; said touch screen illustrating a graphical map an abdomen of an expectant mother.

12. The monitor of claim 11, wherein said at least one processor is configured to receive through said touch screen a location of said at least one transducer on said abdomen of the expectant mother.

13. The monitor of claim 10, wherein a location of said at least one transducer on the abdomen of an expectant mother at which said fetal heartbeat signal was detected is utilized by said at least one processor for future guiding towards the detection of said fetal heartbeat signal.

14. The monitor of claim 1, wherein said at least one processor and said communication module are components included in a single computerized device selected from a group consisting of: (a) smartphone; (b) tablet; (c) laptop; (d) desktop; and (e) mobile phone.

15. The monitor of claim 1, wherein said guidance is selected from the group consisting of guidance based on data from previous searches of fetal heartbeat signals, guidance that takes into consideration time passed from the previous searches, guidance based on a position of the fetus, indication when said fetal heartbeat signal is found, instruction not to move said at least one Doppler transducer when the fetal heartbeat signal is detected, alert when a surface is attached to said at least one transducer, alert when speed of movement of said at least one transducer is too high to acquire the fetal heartbeat signal, alert when pressure applied on said at least one transducer is too low to acquire the fetal heartbeat signal.

16. The monitor of claim 1, wherein a location of said at least one Doppler transducer in which said fetal heartbeat signal was detected is stored by said at least one processor.

17. The monitor of claim 16, wherein said at least one processor is configured to determine at least one of: a change in the position of said fetus, whether said fetus has flipped to a turn down position, whether said fetus is under stress and the well-being of said fetus by comparing a location of said fetal heartbeat signal to said stored location of said at least one Doppler transducer in which said fetal heartbeat signal was previously detected.

18. The monitor of claim 1, further comprising at least one pressure sensing module, to detect pressure of an abdomen of an expectant mother on said at least one Doppler transducer.

19. The monitor of claim 18, wherein said at least one pressure sensing module comprises a plurality of pressure sensors to sense the directionality of the pressure of the abdomen on said at least one Doppler transducer.

20. The monitor of claim 1, further comprising an accelerometer to identify a speed of motion of said at least one Doppler transducer.
